(19) United States
(12) Patent Application Publication
(10) Pub. No.: US 2020/0370067 A1
(43) Pub. Date: Nov. 26, 2020

(54) METHOD TO IDENTIFY AND VALIDATE GENOMIC SAFE HARBOR SITES FOR TARGETED GENOME ENGINEERING

(71) Applicant: UNIVERSITY OF WASHINGTON, Seattle, WA (US)

(72) Inventors: Raymond J. MONNAT, JR., SEATTLE, WA (US); Blake T. HOVDE, SEATTLE, WA (US); Stefan PELLENZ, SEATTLE, WA (US); Michael PHELPS, SEATTLE, WA (US)

(73) Assignee: UNIVERSITY OF WASHINGTON, SEATTLE, WA (US)

(21) Appl. No.: 16/880,877

(22) Filed: May 21, 2020

Related U.S. Application Data
(60) Provisional application No. 62/850,885, filed on May 21, 2019.

(51) Int. Cl. C12N 15/85; C12N 9/22; C12N 15/90; C12Q 1/686

(52) U.S. Cl. CPC C12N 15/85; C12N 9/22; C12N 15/907; C12N 2800/80; C12N 2310/141; C12N 2310/3519; C12Q 1/686

(57) ABSTRACT

Compositions, targeting reagents, modified cells, nucleic acid molecules, systems, and methods for identifying and selecting genomic safe harbor sites for transgene insertion and other genome engineering applications. These materials and methods can be used to develop desired genome engineering applications, such as transgene insertion and expression or genome modification, that take into account the application-specific needs for safety, functional silence, and accessibility and other factors that vary with a desired application's goals and target population. Representative examples of desired genome engineering applications include, but are not limited to, transgene insertion, such as therapeutic transgene insertion, functional gene editing, gene or chromosomal location-specific structural modification, cell marking, gene activation, and/or gene repression. The desired targeting application may act on the site itself to modify it, for example, or to facilitate insertion of a transgene that, upon expression, could lead to gene activation, repression or further modification.

This application claims benefit of U.S. provisional patent application No. 62/850,885, filed May 21, 2019, the entire contents of which are incorporated by reference into this application.

### ACKNOWLEDGEMENT OF GOVERNMENT SUPPORT

This invention was made with government support under Grant Nos, R01 CA196882, T32 HG000035, and CA133831, awarded by the National Institutes of Health. The government has certain rights in the invention.

### REFERENCE TO A SEQUENCE LISTING SUBMITTED VIA EFS-WEB

The content of the ASCII text file of the sequence listing named “UW69USU1_seq” which is 32 kb in size was created on May 21, 2020, and electronically submitted via EFS-Web herewith the application is incorporated herein by reference in its entirety,

### BACKGROUND

Many human genome engineering applications require the introduction and stable integration of transgenes into host cells. For applications that do not require precise targeting of an existing gene or locus (e.g., to introduce or modify an endogenous gene, allele, or regulatory element), a common strategy is to target transgene integration to one of a small number of chromosomal “safe harbor” sites (SHS) for expression, presumably without disrupting the expression of adjacent or more distant genes. These putative SHS play an increasingly important role in developing effective gene therapies; in the investigation of gene structure, function, and regulation; and in cell-based biotechnology.

The most widely used of the putative human SHS, the AAVS1 site on chromosome 19q, was initially identified as a site for recurrent adeno-associated virus insertion, (1; numbers in parentheses correspond to references listed at end of Detailed Description, below). Other potential SHS have been identified on the basis of DNA sequence homology, with sites first identified in other species (e.g., the human homolog of the permissive murine Rosa26 locus (2)) or among the growing number of human genes that appear non-essential under some circumstances, (3,4) One putative SHS of this latter type is the CCR5 chemokine receptor gene, which, when disrupted, confers resistance to human immunodeficiency virus infection. (5) Additional potential genomic SHS have been identified in human and other cell types on the basis of viral integration site mapping (6-8) or gene-trap analyses, as was the original murine Rosa26 locus. (9)

The nature of human SHS identified to date, together with a set of desirable general properties for any SHS, have progressively refined the criteria used to assess the SHS potential of additional sites in the human genome. The first systematic list of SHS criteria grew from early gene therapy trials using viral vectors, most notably for the hemoglobinopathies. (8, 10) These included plausible criteria from first principles, for example location outside of transcriptional units and ultra-conserved regions and from 50-300 kb away from the 5′ ends of genes, cancer-related genes, and micro RNAs, (8, 10) This list was subsequently expanded to include additional, less well-defined criteria such as the exclusion of cell type or lineage-specific essential genes and regulatory RNAs (e.g., long non-coding RNAs), and of cell type-specific, topologically defined nuclear domains (TADS) that have been associated with cancer gene chromatin structure or expressions. Chromatin epigenetic profiles (e.g., of a combination of H3K27 methylation and acetylation marks) have also been used to signal the potential for both high efficiency targeting and persistent transgene expression. (11) All of these criteria depend heavily upon context: cell type and lineage, tissue specificity of gene expression (12,13), and intended application. These considerations identify additional criteria by which to assess potential SHS for use as part of specific gene editing or engineering applications. (11)

There remains a need to expand the number of potentially useful SHS, particularly human SHS, and for methods to validate such sites and select appropriate sites for the development of new types of clinical applications.

### SUMMARY

Described herein are compositions, targeting reagents, modified cells, nucleic acid molecules, and methods for identifying and selecting genomic safe harbor sites for transgene insertion and other genome engineering applications. These materials and methods can be used to develop desired genome engineering applications, such as transgene insertion and expression or genome modification, that take into account the application-specific needs for safety, functional silence, and accessibility and other factors that vary with a desired application's goals and target population. Representative examples of desired genome engineering applications include, but are not limited to, transgene insertion, such as therapeutic transgene insertion, functional gene editing, gene or chromosomal location-specific structural modification, cell marking, gene activation, and/or gene repression. The desired targeting application may act on the site itself to modify it or to facilitate insertion of a transgene that, upon expression, could lead to gene activation, repression or further modification. Some non-limiting examples of expression, editing, and activation of genes using safe harbor sites described herein are shown in FIG. 4.

Disclosed herein is a method of selecting genomic target sites for a desired genome engineering application. One specific example illustrated here is based on the identification of new human safe harbor sites for genome reagent-specific application. The method is applicable to any sequenced genome for which relevant data exist that allow assessment of the criteria outlined below, In one embodiment, the method comprises: (a) seeding a search matrix with putative genomic target site nucleotide sequences having defined target specificity and degeneracy appropriate for the desired targeting application; (b) searching a specified version of a genome reference sequence to identify sites that share at least 95% identity with potential target sites defined in step (a); and (c) selecting sites identified in (b) for which satisfaction of the following predefined criteria can be determined:
(i) unique in reference genome sequence (no more than 1 site per haploid genome);
(ii) not in copy number-variable region;
(iii) target site does not contain nucleotide sequence or other genomic variation that would impede successful targeting;
(iv) at least 25 kilobases (kb) from an unannotated transcript;
(v) at least 50 kb from a 5′ gene end;
(vi) at least 50 kb from an ultra-conserved genomic region, enhancer, or other noncoding regulatory region;
(vii) at least 50 kb from a replication origin;
(viii) at least 300 kb from any microRNA or other functionally annotated small RNA;
(ix) at least 300 kb from a cancer-related gene.

The seeding of a search matrix with putative genomic target site nucleotide sequences having defined target specificity and degeneracy appropriate for the desired targeting application provides a searchable matrix that includes sites that potentially meet the function criteria required for the desired application. Prior to seeding the matrix, the characteristics of possible target sites are defined based on the known properties of the genome targeting method and associated reagents. In some embodiments, the search matrix comprises a position weight matrix (PWM). A PWM is also known as a position-specific search matrix (PSSM).

The selecting of step (c) comprises identifying sites that can be scored for exhibiting the predefined criteria (i)-(ix). These criteria represent desirable properties of safe harbor sites. In some embodiments, the scoring is unambiguous, meaning that each site is capable of being assigned a score of either + (yes, criterion is met) or − (no, criterion not met).Thus, sites for which satisfaction of the criterion cannot be determined (e.g., insufficient information available to determine whether it would be a +or a −), would not be selected.

In some embodiments, the sites are capable of being assigned one of multiple scores, allowing for a weighting or preference to be given to one or more, or all, of the criteria. In one embodiment, each of the sites are assigned one of 3 scores for each criterion: a score of 2 is assigned where a site satisfies all criteria; a score of 1 is assigned where a site satisfies criteria, though not exhaustively, with one or more criteria being indeterminant or lacking requisite data to be determined: and 0 where a site fails to satisfy one or more criteria. In another embodiment, a score of 2 is assigned for each site that does satisfy the criterion, a score of 1 for a site that does not satisfy the criterion, and a score of 0 for sites for which satisfaction of the criterion is either indeterminant or unknown. These scores can then be summed, and used to rank order potential sites such that higher scores indicate a preference for safety, as discussed further below. In some embodiments, a total score aggregated across all criteria is used to prioritize sites for selection and validation.

Thus, in some embodiments, the selecting of step (c) comprises selecting sites that satisfy at least 1, at least 2, at least 3, at least 4, or at least 5 of the 9 criteria. In some embodiments, at least 6, at least 7, or at least 8 of the criteria are met by the sites to be selected. In some embodiments, the selecting is for sites that satisfy all 9 criteria. In other embodiments, the selecting comprises selecting those sites that have been assigned scores that sum at least 12 over all 9 sites, wherein each site receives a score of 0, 1, or 2 for each criterion. In some embodiments, sites are selected when the sum of assigned scores is at least 13, 14, 15, 16, 17, or 18. Alternatively, depending on the desired application, a different scoring can be applied for criteria of greater concern for the intended use.

In some embodiments, the base composition of the target site sequence, e.g., GC or AT-richness, is desired for certain types of targeting methods or reagents (e.g., triplex-forming oligonucleotides). For some agents, this base composition is more important than an exact sequence. This objective can be specified when seeding the search matrix, and can be used to drive an explicitly defined genomic search for close or perfect target site DNA sequence matches.

In some embodiments, specific subsets of the predefined criteria of (c) above, each of (i) through (ix), can be used to assess the safe harbor potential of genomic target sites. In some embodiments, the method further comprises: (d) ranking the putative genornic target sites selected in step (c) according to the desired targeting application; (e) validating target site presence in a targeted genomic sequence, cleavage efficiency of the site(s), and targeted insertion efficiency and fidelity of the transgene at the identified genomic target sites ranked in step (d); and, optionally, (f) assessing genomic or functional effects of desired genome engineering applications at selected sites to identify sites to be deselected due to off-target effects. In some embodiments, the method further comprises generating a list of genomic target sites selected by the method.

In some embodiments, the ranking of step (d) assigns preference to safety, functional silence, and accessibility, respectively. The assignment of preference can be implemented, for example, by assigning a score of 2 for sites that satisfy all criteria, a score of 1 for sites that do satisfy criteria though with one or more criteria indeterminant or lacking requisite data, and a score of 0 for sites that fail to satisfy one or more criteria. Other scorings can be used to adjust the ranking to give greater weight to certain features of greatest importance to the desired targeting application. In some embodiments, the desired targeting application is therapeutic transgene insertion, functional gene editing, gene or chromosomal location-specific structural modification, cell marking, gene activation, and/or gene repression. For example, therapeutic gene editing to correct a heritable human disease in a child requires that long term safety is paramount. Criteria iv-ix directly address these safety concerns in a general sense, and the aggregate scoring across all 5 of these criteria would lead to a rank ordering of a safe harbor site for use in this context. Criterion (i) (uniqueness) addresses the issue of a specific application in a specific context or individual where only a single copy of the target site is present and mapped in the human genome. ‘Unique’ means a single copy of that sequence identified in the whole genome search.

In a representative, non-limiting example, where the desired targeting application is therapeutic transgene insertion, the ranking would depend on a combined assessment of technical feasibility as represented by criteria (i-iii) and safety criteria represented by criteria (iv-ix). Where the desired targeting application is functional gene editing, the ranking would depend critically on feasibility criteria (i-iii above), as the related criteria are already pre-specified by the genomic location of the gene to be edited, Where the desired targeting application is less restrictive, for example cell marking, activation of another gene located at a different chromosomal position, or the editing of a gene at another chromosomal location, the ranking would depend on a combined assessment of technical feasibility as represented by criteria (i-iii) and safety criteria represented by criteria (iv-ix).

In some embodiments, the ranking of step (d) is based on searching genome browser data. In some embodiments, the genome browser data are aggregated at and obtained from

UCSC Genome Browser and/or Ensembl Genome Browser. In some embodiments, the ranking of step (d) is based on scoring genomic target sites that satisfy the set of predetermined criteria of step (c). In some embodiments, the ranking of step (d) is based on assessment of copy number variation and/or base pair level variation in sites identified in (b). In one representative, non-limiting example, the assessment comprises a survey of human population genomic variation data. Such assessment can be updated over time.

In some embodiments, the validating for site presence and cleavage efficiency of step (e) comprises polymerase chain reaction (PCR) amplification of targeted sites and cleavage testing or DNA sequencing. In some embodiments, the validating of step (e) comprises transgene insertion or modification by homology-dependent recombination (HDR) and/or non-homologous DNA end joining (NHEJ) and/or non-cleavage dependent base editing and/or PRIME editing. In some embodiments, the validating of step (e) comprises transgene expression and/or functional assays for a minimum of 10 cell population doublings to assess stability of transgene insertion and expression. In some embodiments, the assessing of step (f) comprises genomic or functional assessments. In some embodiments, the assessing of step (f) is performed in silica.

Also provided is a method of ranking potential genomic target sites for transgene insertion comprising performing a method described above. Additionally provided is a method of producing a targeting construct for insertion of a transgene into a genomic site. In one embodiment, the method comprises: (a) selecting a genomic targeting site according to a method described herein; and (b) synthesizing a construct comprising the transgene flanked by application-specific 5′ and 3′ regulatory sequences, and target site-specific, transgene-flanking homology dependent sequences having sufficient nucleotide sequence homology or identity with the target site sequence to promote transgene insertion into the target site, or homology-independent repair sequence.

Also provided is a targeting construct produced by the above method for use in a specific application. In some embodiments the construct comprises a transgene defined by its intended use or function, flanked by target site-specific DNA sequences flanking the SHS target site to promote transgene chromosomal integration. In some embodiments, the genomic targeting site of (a) is located on chromosome 2p (SHS229), chromosome 4q (SHS231), or on the short arm of chromosome 2, 5, or X, or on the long arm of chromosome 7, 14, or 17 (SHS253). In some embodiments, the genomic targeting site of (a) has a pre-existing target site that can be cleaved by the homodimeric I-Crel homing endonuclease and its monomerized derivative mCrel. In some embodiments, the genomic targeting site of (a) is selected from the group consisting of the target sites listed in Table 2 (SEQ ID NO: 1-27). In some embodiments, the construct is the construct shown in FIG. 2. In some embodiments, the construct targets human chromosome 4 SHS231 and is selected from the group consisting of: pSH231-EF1-Puro, pSH231-EF1-GFP-HYGRO, pSH231-EF1-RFP-HYGRO, pSH231-EFS-Cas9-BlastR, pSH231-EF1-BLST-Cas9-VPR, pSH231-EF1-BLST-dCas9-VPR, pSH-231-Bx-GFP-031, and pUS2-SH231.

In some embodiments, the insertion of the construct is mediated by a targeting reagent. A targeting reagent is an active agent that is site-specific and serves as a mediator of a defined activity on a target site that, in some embodiments, may involve a third entity, such as a transgene. The targeting reagent is typically a protein, nucleic acid sequence, or nucleoprotein complex, that, upon introduction into a cell, can cleave or otherwise perform a defined activity on a target site to modify that site, including reagents useful in non-cleavage dependent base editing and PRIME editing. In some embodiments, the targeting reagent comprises a homing nuclease, a meganuclease, Cas9, or TALEN that can cleave a specific target site with high efficiency to mutate that site or catalyze transgene insertion.

Described herein is a cell modified by insertion of a targeting construct. In some embodiments, the cell is modified by insertion of a Bxb1 landing-pad at genomic target site SHS231. In some embodiments, the cell is modified by insertion of a targeting construct that is identical to or derived from a targeting construct described herein. In some embodiments, the cell is from a standard cell line, such as, for example, a U-2 OS or RPE1 cell; or from a squamous cell carcinoma cell line, such as, for example, FaDu, UM-SCC-01, SFCI-SCC9 cells;

or from a rhabdomyosarcoma cell line, such as, for example, 381T SH-BlastR-dCas9-VPR, 381T SH-MS2-p65/HSF-BlastR, Rh30 SH MS2-P65/HSF, Rh30 SH-Cas9-BlasR, Rh30 SH-Cpf1, Rh5 SH-BlastR-dCas9-VPR, Rh5 SH-GFP-Hygro, SMSCtr SH VSVG Puro, SMSCtr SH-BlastR-dCas9-VPR, SMSCtr SH-BlastR-MS2-P65/HSF, SMSCtr SH-Cas9-VPR-BlastR, SMSCtr SH-GFP-Hygro, and SMSCtr SH-Puro AttP. In some embodiments, the cell is modified by insertion of a functionally complementing FANCA transgene at genomic target site SHS231.

In some embodiments, the method is implemented on a computer, the computer having one or more processors and a memory storing one or more programs for execution by the one or more processors, the one or more programs including instructions for performing steps (a) to (c). In some embodiments, the seeding of step (a) comprises receiving by the processor instructions to load a target genorne sequence and a list of putative target site sequences, wherein the target genome sequence is specified by a genome browser or other defined genome source files, and wherein the list of putative target site sequences is pre-defined list or generated from an algorithm. In some embodiments, the searching of step (b) comprises receiving by the processor instructions to exclude target sites containing insertions or deletions with respect to the reference sequence. In some embodiments, the selecting of step (c) comprises receiving instructions (i) to identify one or more criteria selected from: copy number variable regions, microRNAs, ultra-conserved regions, replication origins, non-coding regulatory elements, annotated transcripts, unannotated transcripts, and regions of open chromatin, and (ii) to assign a score indicative of the identified criteria.

Also provided herein is a system for selecting genomic target sites for transgene insertion or other desired genome engineering application. In one embodiment, the system comprises a user device comprising a hardware processor that is programmed to perform the method of selecting genomic target sites described herein. Additionally provided is a non-transitory computer-readable medium containing computer executable instructions that, when executed by a processor, cause the processor to perform the method. Such systems and executable instructions are designed to and capable of implementing assessment of the above methods individually or wholly on a defined genome sequence.

The subject genome to be targeted in the methods disclosed herein is typically a mammal, such as a human or veterinary subject. The method is applicable to any sequenced genome for which relevant data exist that allow assessment of the target site selection or assessment criteria outlined herein.

### BRIEF DESCRIPTION OF THE DRAWINGS

FIG. 1. Identification and mapping of new human safe harbor sites (SHS). (A) The canonical mCrel horning endonuclease cleavage site is shown top with twofold symmetric basepair positions shaded (SEQ ID NO: 51). The matrix below summarizes the functional consequences of basepair insertions across the mCrel target site (positions 1-18 of SEQ ID NO: 51) where a value of 1=native site cleavage efficiency and values <0.3 indicate cleavage resistance. Basepairs highlighted with shading indicate either the canonical basepair at that position, or a highly cleavable basepair substitution. (B) Workflow for identifying highly cleavage-sensitive mCrel target sites in the human genome sequence. (C) Physical confirmation and functional verification of two new unique SHS located on chromosomes 2p (SHS229) and 4q (SHS231). A third highly ranked SHS (SHS253) was identified at 6 locations on the short arms of chromosomes 2, 5 and X and the long arms of chromosomes 7, 14 and 17. Asterisks (*) indicate sites where basepair variants have been identified in the mCrel target site in human population genetic data.

FIG. 2. Molecular confirmation of SHS231 homology-dependent editing by three engineering nucleases. The top panel shows the locations of cleavage sites for mCrel, TALEN and CRISPR/Cas9 nucleases centered on the chromosome 4 SHS231 safe harbor site (key shown top right), with the structure of the 1.05 kb repair template shown below. The bottom panel shows independently cloned and sequenced inserts from targeted SHS231 insertions by all 3 nucleases (SEQ ID NO: 28; locus shown corresponds to positions 1-25 and 74-98 of SEQ ID NO: 28). The mCrel targeting experiments used an expression vector that encoded both mCrel and the TREX2 nuclease, and Cas9 targeting was performed using a common guide RNA and either a Cas9 cleavage or nickase. Numbers to the right of each row indicates the number of independent targeting events that were cloned and sequenced.

FIG. 3. Homology-independent engineering of the chromosome 4q SHS231. (A) Strategy for targeted integration of transgene cassettes using NHEJ mediated repair. Triangles represent gRNA target sites on both the genome and repair template. Representative sequences from the 5′ transgene integration site after knockin specific PCR amplification of an integrated transgene (striped arrows: SEQ ID NO: 29). (B) Relative knockin efficiency of a puromycin cassette using homology independent repair (US2-Cas9; NHEJ), and homology directed repair (nCas9, Cas9, mCrel; HDR) at the SHS231 locus, compared to piggybac transposition (PBase). (C) Quantification of crystal violet staining from SHS231 knockin stable cells. Significantly different from HDR SH5231 knockin approaches, P<0.05.

FIG. 4. Stable expression of functional gene editing and gene activation proteins encoded by SHS231 transgenes. (A) Long-term stable GFP expression from a SHS231 integrated transgene in two independent RMS cell lines. (B) Relative Cas9 expression level (cycle threshold: Ct) from a SHS231 integrated Cas9 cassette compared to cells transduced with high titer Cas9 expressing lentivirus or the endogenous expression level of GAPDH. Both SHS231 and lentiviral Cas9 variants were expressed from the human EF1α promoter. (C) Targeted deletion of a 17,188 bp gDNA segment of the PAX3/FOXO1 fusion oncogene in Rh30 RMS cells expressing Cas9 from the SHS231 locus. Dual gRNA target sites (triangles) and deletion PCR primer sites (striped arrows) are identified. (D) Demonstration of endogenous MYF5 gene activation with SHS231 expressed dCas9-VPR and Cas9-VPR transgenes. Gene activation was achieved by targeting full length (20 bp) or truncated (14 bp) gRNAs (white, black, and striped triangles) to the promoter region of the MYF5 gene.

FIG. 5. SHS231 endonuclease and repair template constructs. (A) Details of the SHS231 locus with homology dependent (HDR) and homology independent (NHEJ) gRNA target sites identified along with the location of repair template homology arms (dashed boxes). (B) Features of the endonuclease expression and repair template vectors are identified in the legend. The gRNA stippling and shading correspond to target sites in the safe harbor locus and in repair template homology arms.

FIG. 6. Restriction site analysis from HDR integration of a loxP cassette into the SHS229 and SHS253 loci.

FIG. 7. Workflow illustration of human genomic safe harbor site region with inclusion and exclusion criteria and zones.

FIG. 8. Screenshot image of exemplary selections for identifying criteria for inclusion and exclusion per steps 1 and 2 of the workflow illustrated in FIG. 7, as viewed when interfacing with UCSC Genome Browser.

FIG. 9. Screenshot image of exemplary selections for identifying criteria for inclusion and exclusion per steps 3 and 4 of the workflow illustrated in FIG. 7, as viewed when interfacing with UCSC Genome Browser.

### DETAILED DESCRIPTION

The methods described herein greatly expand the number of useful human SHS, and provide a means to identify sites that are more suitable than the canonical sites in current use Moreover, these methods enable the identification of a multiplicity of SHS and the ability to target by genome arm. To develop and explore these methods, the human genome was searched for target-site regions containing target sites for three classes of genome-editing nuclease in close proximity. The 35 sites identified in this way were then assessed for SHS potential using eight different genomic criteria in parallel with the existing human AAVS1, ROSA26, and CCR5 sites. Several potential new SHS were experimentally characterized to demonstrate functional competence for efficient, targeted transgene insertion and expression in different human cell types. These 35 new human SHS, located on 16 different human chromosomes and 23 chromosome arms, including both arms of the human X chromosome, provide an expanded list of potential human SHS for targeted transgene insertion to enable basic science as well as clinical applications. A representative subset of these new sites has been further experimentally validated, and experimental evidence is provided for successful targeting, transgene insertion, and persistent expression of selectable, scorable, or functionally active proteins.

### Definitions

All scientific and technical terms used in this application have meanings commonly used in the art unless otherwise specified. As used in this application, the following words or phrases have the meanings specified.

As used herein, the term “appropriate” in the context of “nucleotide sequences having target specificity and degeneracy appropriate for the desired targeting application” refers to a corresponding level of complementarity and/or nucleotide sequence identity to allow for efficient targeting with transgene insertion. Appropriate for the desired targeting application means that a site is permissive of general features that are consistent with the desired activity.

As used herein, “application-specific 5′ and 3′ regulatory sequences” refers to promoter and RNA synthesis and degradation sequences that mediate regulated expression of the transgene in the context of the insertion site.

As used herein, the term “comprising” is intended to mean that the compositions and methods include the recited elements. but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the disclosure herein.

As used herein, the terms “nucleic acid sequence” or “polynucleotide” refers to nucleotides of any length which are deoxynucleotides (i.e. DNAs), or derivatives thereof: ribonucleotides (i.e. RNAs) or derivatives thereof; or peptide nucleic acids (PNAs) or derivatives thereof. The terms include, without limitation, single-stranded, double-stranded, or multi-stranded DNA or RNA, genomic DNA, cDNA, DNA-RNA hybrids, oligonucleotides (oligos), or other natural, synthetic, modified, mutated or non-natural forms of DNA or RNA,

MicroRNAs, or “miRNAs”, or “miRs”, are short, non-coding RNAs that regulate gene expression by post-transcriptional regulation of target genes.

“Short hairpin RNAs” or “shRNAs” are synthetic or non-natural RNA molecules. shRNA refers to RNA with a tight hairpin turn used to silence (via RNA interference or RNAi) target gene expression in a cell. An shRNA is typically delivered via an expression vector such as a DNA plasmid or via viral vectors.

The term “vector” refers to, without limitation, a recombinant genetic construct or plasmid or expression construct or expression vector that retains the ability once transfected or transduced into a cell to express a transgene upon integration into the chromosome or upon stable maintenance within the cell.

The term “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, boundary or insulator elements and introns. Expression control elements may be, without limitation, constitutive, inducible, repressible, or tissue-specific. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. An “enhancer” is a region of DNA that can be bound by activating proteins to increase the likelihood or frequency of transcription. Non-limiting exemplary enhancers and posttranscriptional regulatory elements include the CMV enhancer and WPRE.

The term “multicistronic” or “polycistronic” or “bicistronic” or tricistronic” refers to mRNA with multiple, i.e., double or triple coding areas or exons, and as such will have the capability to express from mRNA two or more, or three or more, or four or more, etc., proteins from a single construct. Multicistronic vectors simultaneously express two or more separate proteins from the same mRNA. The two strategies most widely used for constructing multicistronic configurations are through the use of 1) an IRES or 2) a 2A or 2P self-cleaving site. An “IRES” refers to an internal ribosome entry site or portion thereof of viral, prokaryotic, or eukaryotic origin which are used within polycistronic vector constructs, In some embodiments, an IRES is an RNA element that allows for translation initiation in a mRNA cap-independent manner. The term “self-cleaving peptides” or “sequences encoding self-cleaving peptides” or “2A or 2P self-cleaving site” refer to linking sequences which are used within vector constructs to incorporate sites to promote ribosomal skipping followed by nascent polypeptide self-cleavage at the self-cleaving site and thus to generate two polypeptides from a single promoter. Such self-cleaving peptides include without limitation, T2A, and P2A peptides or sequences encoding the self-cleaving peptides.

The term “substantially complementary,” when used to define either amino acid or nucleic acid sequences, means that a particular sequence, for example, an oligonucleotide sequence, is substantially identical in sequence to the sequence referenced. As such, typically the sequences will be highly complementary to the “target” sequence, and will have no more than 1, 2, 3, 4, 5, 6, 7, 8, 9, or 10 base pair or amino acid differences throughout the sequence. In a typical embodiment, the sequences will exhibit at least 95% complementarity to the target sequence. In many instances, it may be desirable for the sequences to be exact matches, i.e. be completely complementary to the sequence to which the nucleic acid specifically binds, and therefore have zero mismatches along the complementary stretch, or have no amino acid residue differences. As such, highly complementary sequences will typically bind quite specifically to the target sequence region and will therefore be highly efficient in targeting an intended biological or biochemical activity to the target sequence.

Substantially complementary nucleic acid sequences will be greater than about 90 percent complementary (or ‘% exact-match’) to the corresponding target sequence to which the nucleic acid or protein specifically binds. In certain aspects, as described above, it will be desirable to have even more substantially complementary nucleic acid sequences for use in the practice of the invention, and in such instances, the nucleic acid sequences will be greater than 95 percent complementary to the corresponding target sequence to which the nucleic acid specifically binds, up to and including 96%, 97%, 98%, 99%, and even 100% exact match complementary to the target to which the designed nucleic acid specifically binds.

“Homology” or “identity” or “similarity” refers to position-specific sequence identity or chemical similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology between sequences is a function of the number of matching identical or homologous, chemically similar elements shared by sequences at equivalent amino acid or basepair positions in aligned sequences. An “unrelated” or “non-homologous” sequence shares less than 40% identity, or alternatively less than 25% identity, with one of the sequences of disclosed herein.

Percent similarity or percent complementary of any of the disclosed sequences may be determined, for example, by comparing sequence information using one of the suite of BLAST algorithms and search engines available via the NCBI (National Center for Biotechnology Information) at blast.ncbi.nlm.nih.gov/Blast.cgi. BLAST versions allow the pre-specification of search parameters and tolerances for gaps and mismatches/non-identities on both protein and nucleotide sequences (Altschul, S. F., Gish, W., Miller, W., Myers, E. W. & Lipman, D. J. (1990) “Basic local alignment search tool.” J. Mol. Biol. 215:403-410).

“Nucleotide sequence” refers to a heteropolyrner of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides, to provide a sequence which is capable of specifically binding to a target molecule or act as an antisense construct to alter, reduce, or inhibit the biological activity of the target.

As used herein, the terms “protein”, “peptide”, and “polypeptide” refer to amino acid subunits, amino acid analogs, or peptidomimetics. The subunits are typically linked by peptide bonds. In another aspect, the subunit may be linked by other bonds, e.g., ester, ether, etc. As used herein the term “amino acid” refers to either natural and/or unnatural or synthetic amino acids.

As used herein, the term “recombinant expression system” or “recombinant expression vector” refers to a genetic construct for the expression of certain genetic material formed by recombination.

When the disclosure herein relates to a small molecule, polypeptide, protein, polynucleotide, nucleic acid, oligonucleotide, antisense, or miRNA, an equivalent or a biologically equivalent of such is intended within the scope of this disclosure, As used herein, the term “biological equivalent thereof” is intended to be synonymous with “equivalent thereof” when referring to a reference small molecule, polypeptide, protein, polynucleotide, nucleic acid, oligonucleotide, antisense, or miRNA even those reference molecules having minimal homology while still maintaining desired structure or functionality. Unless specifically recited herein, it is contemplated that any nucleic acid, polynucleotide, oligonucleotide, antisense, miRNA, polypeptide, or protein mentioned herein also includes equivalents thereof. For example, an equivalent intends at least 70% homology or identity, or at least 80% homology or identity, or at least about 85%, or at least about 90%, or at least about 95%, or alternatively 98% percent homology or identity in order to capture and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

In some embodiments disclosed herein, the polypeptide and/or polynucleotide sequences are provided herein for use in gene and protein transfer and expression techniques described below. Such sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polynucleotide or polypeptide sequences are provided as examples of particular embodiments. Modifications may be made to the amino acid sequences by using alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a polymerase chain reaction, or the enzymatic cleavage of a polynucleotide by a ribozyme.

As used herein, “treating” or “treatment” of a condition or disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results.

As used herein, a cancer-related gene is a gene known to be associated with cancer. One listing of such genes is the ‘Catalogue of Somatic Mutations in Cancer’ database (‘COSMIC’) at the Sanger Institute: cancer.sanger.ac.uk/census. For example, COSMIC version 89 lists 723 genes at present, in GRCh38/hg38 coordinates.

As used herein, the term “isolated” means that a naturally occurring DNA fragment, DNA molecule, coding sequence, or oligonucleotide is removed from its natural environment, or is a synthetic molecule or cloned product. Preferably, the DNA fragment, DNA molecule, coding sequence, or oligonucleotide is purified, i.e., essentially free from any other DNA fragment, DNA molecule, coding sequence, or oligonucleotide and associated cellular products or other impurities.

The term “cell” as used herein refers to either a prokaryotic or eukaryotic cell, optionally obtained from a subject or a commercially available source. Cells treated, transfected, transformed, transduced or otherwise in contact with compositions and/or nucleic acid molecules disclosed herein, include without limitation, cells of a human, non-human animal, mammal, or non-human mammal, including without limitation, cells of murine, canine, or non-human primate species.

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects.

As used herein, “a” or “an” means at least one, unless clearly indicated otherwise.

As used herein, to “prevent” or “protect against” a condition or disease means to hinder, reduce or delay the onset or progression of the condition or disease.

The term “encode” as it is applied to nucleic acid sequences refers to a polynucleotide which is said to “encode” a polypeptide, an mRNA, or an effector RNA if, in its native state or when manipulated by methods well known to those skilled in the art, can be transcribed and/or translated to produce the cognate effector RNA, mRNA, or polypeptide and/or a fragment thereof. The antisense strand is the complement of such a nucleic acid, and the encoding sequence can be deduced therefrom.

As used herein, the term “expression” or “gene expression” refers to the process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

As used in the description of the invention and the appended claims, the singular forms “a,” “an” and “the” are intended to include the plural forms as well, unless the context clearly indicates otherwise.

The term “about,” as used herein when referring to a measurable value such as an amount, level or concentration, for example and without limitation, is meant to encompass variations of 20%, 10%, 5%, 1%, 0.5%, or even 0.1% of the specified amount, or fold differences in levels of a quantifiable comparison with a standard or control or reference material, such as 1-fold, 2-fold, 3-fold, 4-fold . . . 10-fold, 100-fold, etc. of the specified level of comparison.

The terms “acceptable,” “effective,” or “sufficient” when used to describe the selection of any components, ranges, dose forms, etc. disclosed herein intend that said component, range, dose form, etc. is suitable for the disclosed purpose.

### Methods of Identifying and Selecting Safe Harbor Sites

Disclosed herein is a method of genome engineering. In one aspect, provided is a method of selecting genomic target sites for a desired genome engineering application. In one embodiment, the method comprises: (a) seeding a search matrix with putative genomic target site nucleotide sequences having defined target specificity and degeneracy appropriate for the desired targeting application; (b) searching a specified version of a genome reference sequence to identify sites that share at least 95% identity with potential target sites defined in step (a); and (c) selecting sites identified in (b) for which satisfaction of the following predefined criteria can be determined:
(i) unique in reference genome sequence (no more than 1 site per haploid genome);
(ii) not in a copy number-variable (genome) region;
(iii) target site does not contain nucleotide sequence or other genomic variation that would impede successful targeting;
(iv) at least 25 kilobases (kb) from an unannotated transcript;
(v) at least 50 kb from a 5′ gene end;
(vi) at least 50 kb from an ultra-conserved genomic region, enhancer, or other noncoding regulatory region;
(vii) at least 50 kb from a replication origin;
(viii) at least 300 kb from any microRNA or other functionally annotated small RNA;
(ix) at least 300 kb from a cancer-related gene.

The seeding of a search matrix with putative genomic target site nucleotide sequences having defined target specificity and degeneracy appropriate for the desired targeting reagent and application provides a searchable matrix that includes sites that potentially meet the function criteria required for the desired application. The seed sequences are driven by the properties of the targeting agent. Prior to seeding the matrix, the characteristics of possible target sites are defined based on the known properties of the genome targeting method and associated reagents. For example, one can structure the search for new SHS by identifying matches in the target genome to sequences of a desired endonuclease, such as the rare cutting human LAGLIDADG family homing endonuclease mCrel. This collection of all possible sites that could potentially meet the desired requirements can then be assessed for whether the sites potentially meet functional criteria, such as a high level of cleavage specificity. In one example described herein, the number of sites meeting the functional criterion have mCrel target-site variants predicted to be cleaved with at least 90% of the efficiency of the native mCrel site was 128. These 128 candidate target sites were then seeded into a search matrix. A BLAST search can then be performed with these candidate target sites using desired criteria for high-quality matches, length, etc. as appropriate to the desired targeting application,

In some embodiments, the search matrix comprises a position weight matrix (PWM). A PWM is also known as a position-specific search matrix (PSSM). These matrices are constructed from experiments in which each base pair position in a target site sequence is altered sequentially to represent the three possible single base changes, in conjunction with functional assessment of the cleavage sensitivity and specificity of each variant. Search matrices and accompanying experimental data can be further expanded to include the consequences of additional types of genomic variation (e.g., insertions, deletions and >1 bp alterations). The search matrix takes into account the known target site specificity and sequence of a specified genome editing gene editing technology, methodology or reagent, and the functional consequences of changes at each base pair position in that target site. An example is the known target/cleavage site of the homodimeric I-Crel homing endonuclease and its monomerized derivative mCrel.

The searching of step (b) comprises searching a specified version of a genome reference sequence to identify sites that share at least 95% identity with potential target sites defined in step (a). The specified version is typically both species-specific (e.g., human or other species of interest) and an identified version of a genome reference sequence. The selection of the most appropriate version of a genome reference sequence can be significant in order to work with the most cross-referenced data sets with respect to the desired targeting application. In some embodiments, the genome reference sequence is a human genome reference sequence. In other embodiments, the genome reference sequence is a murine, bovine, ovine, porcine, equine, avian, piscine, or other genome.

The selecting of step (c) comprises identifying sites that can be scored for exhibiting the predefined criteria (i)-(ix). These criteria represent desirable properties of safe harbor sites. In some embodiments, the scoring is unambiguous, meaning that each site is capable of being assigned a score of either + (yes, criterion is met) or − (no, criterion not met).Thus, sites for which satisfaction of the criterion cannot be determined (e.g., insufficient information available to determine whether it would be a + or a −), would not be selected or would be ranked lower.

In some embodiments, the sites are capable of being assigned one of multiple scores, allowing for a weighting or preference to be given to one or more, or all, of the criteria. In one embodiment, each of the sites are assigned one of 3 scores for each criterion: a score of 2 is assigned where a site satisfies all criteria; a score of 1 is assigned where a site satisfies criteria, though not exhaustively, with one or more criteria being indeterminant or lacking requisite data to be determined: and 0 where a site fails to satisfy one or more criteria. In another embodiment, a score of 2 is assigned for each site that does satisfy a particular criterion, a score of 1 for a site that does not satisfy the criterion, and a score of 0 for sites for which satisfaction of the criterion is either indeterminant or unknown. These scores can then be summed, and used to rank order potential sites such that higher scores indicate a preference for safety, as discussed further below. In some embodiments, a total score aggregated across all criteria is used to prioritize sites for selection and validation.

Thus, in some embodiments, the selecting of step (c) comprises selecting sites that satisfy at least 1, at least 2, at least 3, at least 4, or at least 5 of the 9 criteria. In some embodiments, at least 6, at least 7, or at least 8 of the criteria are met by the sites to be selected. In some embodiments, the selecting is for sites that satisfy all 9 criteria. In other embodiments, the selecting comprises selecting those sites that have been assigned scores that sum at least 12 over all 9 sites, wherein each site receives a score of 2, 1, or 0 for each criterion. In some embodiments, sites are selected when the sum of assigned scores is at least 13, 14, 15, 16, 17, or 18. Alternatively, depending on the desired application, a different scoring can be applied for criteria of greater concern for the intended use.

In some embodiments, the base composition of the target site sequence, e.g., GC- or AT-richness, is desired for certain types of targeting methods or reagents (e.g., triplex-forming oligonucleotides). For some agents, this base composition is more important than an exact sequence. This objective can be specified when seeding the search matrix, and can be used to drive an explicitly defined genomic search for close or perfect target site DNA sequence matches.

Whether a target site contains nucleotide sequence or other genomic variation that would impede successful targeting can be indicated by absence of a potential target site from the list of allowable sites as defined in (a) above. This determination can be predefined given the known biochemical or physical properties of the targeting reagent in conjunction with pre-existing data on what degrees of tolerance there are from the canonical sequence that would indicate whether targeting would or would not occur, or might be inefficient. A discussion of basepair variation can be found in the example below, in which it was possible to assess all target sites across a population of individuals to identify basepair variation in a small subset of sites in some individuals. This analysis revealed that almost all sites were useable in almost all individuals.

In some embodiments, specific subsets of the predefined criteria of (c) above, each of (i) through (ix), can be used to assess the safe harbor potential of genomic target sites. In some embodiments, the method further comprises:
(d) ranking the putative genomic target sites selected in step (c) according to the desired targeting application;
(e) validating target site presence in a targeted genomic sequence, cleavage efficiency of the site(s), and targeted insertion efficiency and fidelity of the transgene at the identified genomic target sites ranked in step (d); and, optionally,
(f) assessing genomic or functional effects of desired genome engineering applications at selected sites to identify sites to be deselected due to off-target effects.

In some embodiments, the ranking of step (d) assigns preference to safety, functional silence, and accessibility, respectively. If all are satisfied at a minimum, there may still be nuances or preferences, e.g., related to a cell type, tissue or equivalent that might allow a further sorting of nominally equivalent sites. The assignment of preference can be implemented, for example, by assigning a score of 2 for sites that satisfy a given criterion, a score of 1 for sites that meet in part given criteria, and a score of 0 for sites for which the criteria are not met or the requisite data are not available. Other scorings can be used to adjust the ranking to give greater weight to certain features of greatest importance to the desired targeting application. In some embodiments, the desired targeting application is therapeutic transgene insertion, functional gene editing, gene or chromosomal location-specific structural modification, cell marking, gene activation, and/or gene repression. For example, therapeutic gene editing to correct a heritable human disease in a child requires that long term safety is paramount. Criteria iv-ix directly address these safety concerns in a general sense, and the aggregate scoring across all 5 of these criteria would lead to a rank ordering of a safe harbor site for use in this context. Criterion (i) (uniqueness) addresses the issue of a specific application in a specific context or individual where only a single copy of the target site is present and mapped in the human genome. ‘Unique’ means a single copy of that sequence identified in the whole genome search.

In a representative, non-limiting example, where the desired targeting application is therapeutic transgene insertion, the ranking would depend on a combined assessment of technical feasibility as represented by criteria (i-iii) and safety criteria represented by criteria (iv-ix). Where the desired targeting application is functional gene editing, the ranking would depend critically on feasibility criteria (i-iii above), as the related criteria are already pre-specified by the genomic location of the gene to be edited, Where the desired targeting application is less restrictive, for example cell marking, activation of another gene located at a different chromosomal position, or the editing of a gene at another chromosomal location, the ranking would depend on a combined assessment of technical feasibility as represented by criteria (i-iii) and safety criteria represented by criteria (iv-ix).

In some embodiments, the ranking of step (d) is based on searching genome browser data, In some embodiments, the genome browser data are aggregated at and obtained from UCSC Genome Browser and/or Ensembl Genome Browser. In some embodiments, the ranking of step (d) is based on scoring genomic target sites that satisfy the set of predetermined criteria of step (c). In some embodiments, the ranking of step (d) is based on assessment of copy number variation and/or base pair level variation in sites identified in (b). In one representative, non-limiting example, the assessment comprises a survey of human population genomic variation data. The survey of human population genomic variation data can be updated over time. The survey of target site-specific human population genomic variation data identifies variation known to render targeting of that variant site either resistant or refractory to targeted modification by a specified genome editing reagent. For example, a common insertion site sequence was discovered near SHS231. With such foreknowledge, this can be accommodated and not reduce editing efficiency.

In some embodiments, the validating for site presence and cleavage efficiency of step (e) comprises polymerase chain reaction (PCR) amplification of targeted sites and cleavage testing or DNA sequencing. In some embodiments, the validating of step (e) comprises transgene insertion or modification by homology-dependent recombination (HDR) and/or non-homologous DNA end joining (NHEJ). In some embodiments, the validating of step (e) comprises transgene expression and/or functional assays for a minimum of 10 cell population doublings to assess stability of transgene insertion and expression. In some embodiments, the assessing of step (f) comprises genomic or functional assessments. In some embodiments, the assessing of step (f) is performed in silica. This step allows for exclusion of sites with a demonstrable or too high a level of off-target activity.

Also provided is a method of ranking potential genomic target sites for transgene insertion comprising performing a method described above. Additionally provided is a method of producing a targeting construct for insertion of a transgene into a genomic site. In one embodiment, the method comprises:
(a) selecting a genomic targeting site according to a method described herein; and
(b) synthesizing a construct comprising the transgene flanked by application-specific 5′ and 3′ regulatory sequences, and target site-specific, transgene-flanking homology dependent sequences having sufficient nucleotide sequence homology or identity with the target site sequence to promote transgene insertion into the target site, or homology-independent repair sequence.

### Constructs and Cells for Targeting Safe Harbor Sites

Provided herein are nucleic acid constructs, including endonuclease expression constructs, repair template constructs, and targeting constructs for use in a specific genome engineering application. The constructs include, but are not limited to, DNA cassettes for introducing targeted mutations into human genes, and for activating or repressing gene expression. In some embodiments, the constructs can further include elements for expressing fluorescent reporters (GFP, RFP), the VSVG envelope protein, and for integration of integrase attP landing pads, for example. A “targeting construct” is capable of transferring gene sequences to a target site. In some embodiments the construct comprises a transgene defined by its intended use or function, flanked by target site-specific DNA sequences flanking the SHS target site to promote transgene chromosomal integration.

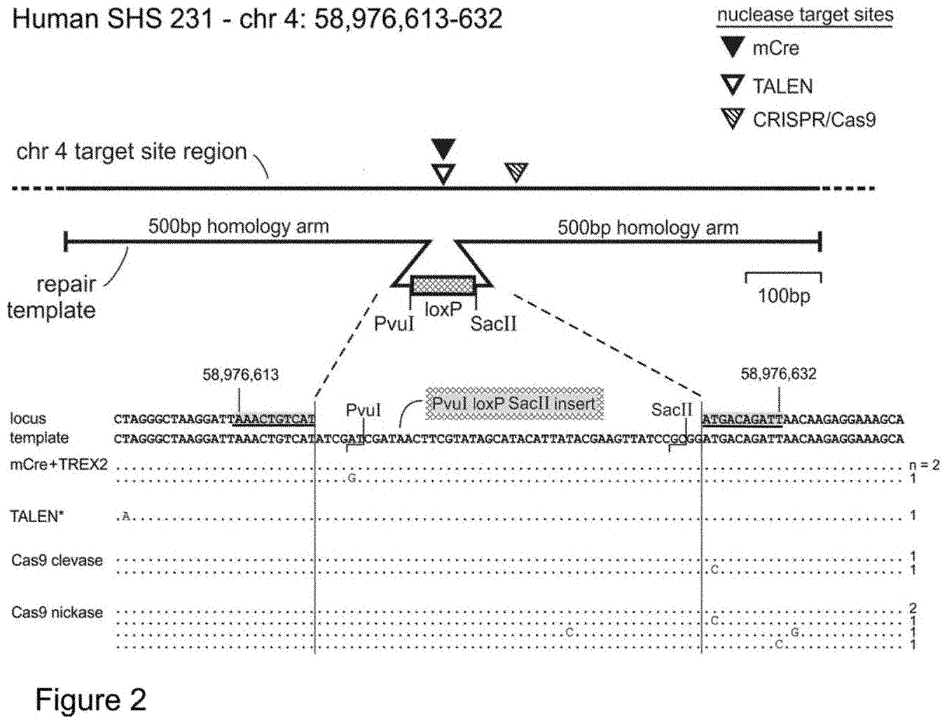

In some embodiments, the genomic targeting site of (a) is located on chromosome 2p (SHS229), chromosome 4q (SHS231), or on the short arm of chromosome 2, 5, or X, or on the long arm of chromosome 7, 14, or 17 (SH5253) In some embodiments, the genomic targeting site of (a) has a pre-existing target site that can be cleaved by the homodimeric I-Crel homing endonuclease and its monomerized derivative mCrel. In some embodiments, the genomic targeting site of (a) is selected from the group consisting of the targeting sites listed in Table 2 (SEQ ID NO: 1-27). In some embodiments, the construct is the construct shown in FIG. 2. In some embodiments, the construct targets human chromosome 4 SHS231 and is selected from the group consisting of: pSH231-EF1-euro, pSH231-EF1-GFP-HYGRO, pSH231-EF1-RFP-HYGRO, pSH231-EFS-Cas9-BlastR, pSH231-EF1-BLST-Cas9-VPR, pSH231-EF1-BLST-dCas9-VPR, pSH231-Bx-GFP-C31, and pUS2-SH231. Representative constructs are listed in Table 5.

In some embodiments, the insertion of the construct is mediated by a targeting reagent. A targeting reagent is an active agent that is site-specific and serves as a mediator of a defined activity on a target site that, in some embodiments, may involve a third entity, such as a transgene. The targeting reagent is typically a protein, nucleic add sequence, or nucleoprotein complex, that, upon introduction into a cell, can cleave or otherwise perform a defined activity on a target site to modify that site. In some embodiments, the targeting reagent comprises a horning nuclease, a meganuclease, Cas9, or TALEN that can cleave a specific target site with high efficiency to mutate that site or catalyze transgene insertion.

Also provided is a cell modified by insertion of a targeting construct. In some embodiments, the cell is modified by insertion of a Bxb1 recombinase landing-pad at genomic target site SHS231. In some embodiments, the cell is modified by insertion of a targeting construct that is identical to or derived from a targeting construct described herein. In some embodiments, the cell is from a standard cell line, such as, for example, a U-2 OS or RPE1 cell; or from a squamous cell carcinoma cell line, such as, for example, FaDu, UM-SCC-01, SFCI-SCC9 cells; or from a rhabdomyosarcoma cell line, such as, for example, 381T SH-BlastR-dCas9-VPR, 381T SH-M2-p65/HSF-BlastR, Rh30 SH MS2-P65/HSF, Rh30 SH-Cas9-BlasR, Rh30 SH-Cpf1, Rh5 SH-BlastR-dCas9-VPR, Rh5 SH-GFP-Hygro, SMSCtr SH VSVG Puro, SMSCtr SH-BlastR-dCas9-VPR, SMSCtr SH-BlastR-MS2-P65/HSF, SMSCtr SH-Cas9-VPR-BlastR, SMSCtr SH-GFP-Hygro, and SMSCtr SH-Puro AttP. In some embodiments, the cell is modified by insertion of a functionally complementing FANCA transgene at genomic target site SHS231. Other examples of cell lines include, but are not limited to, HEK293T or Hela cells.

### Systems

In one aspect, described herein is a computer implemented method for selecting genomic target sites for a desired genome engineering application. In some embodiments, the system comprises a device having one or more processors and a memory storing one or more programs for execution by the one or more processors, the one or more programs including instructions for: (a) seeding a search matrix with putative genomic target site nucleotide sequences having defined target specificity and degeneracy appropriate for the desired genome engineering application; and (b) searching a specified version of a genome reference sequence to identify sites that share at least 95% identity with potential target sites defined in step (a). This identity refers to identity at the individual base pair level, with no gaps or additions with respect to the query sequence. Length variation is avoided by either excluding or disfavoring insertion or deletion variants.

The one or more programs further include instructions for: (c) selecting sites identified in (b) for which satisfaction of the following predefined criteria can be determined:
(i) unique in the reference genome sequence (no more than 1 site per haploid genome);
(ii) not in copy number-variable region;
(iii) target site does not contain nucleotide sequence or other genomic variation that would impede successful targeting;
(iv) at least 25 kilobases (kb) from an unannotated transcript;
(v) at least 50 kb from a 5′ gene end;
(vi) at least 50 kb from an ultra-conserved genomic region, enhancer, or other noncoding regulatory region;
(vii) at least 50 kb from a replication origin;
(viii) at least 300 kb from any microRNA or other functionally annotated small RNA;
(ix) at least 300 kb from a cancer-related gene.

In some embodiments, the one or more programs further include instructions for:
(d) ranking the putative genomic target sites selected in step (c) according to the desired genome engineering application;
(e) optionally, validating target site presence in a targeted genomic sequence, cleavage efficiency of the site(s), and targeted insertion efficiency and fidelity of the transgene at the identified genomic target sites ranked in step (d), or analyzing information obtained from experimental validation; and, optionally,
(f) assessing genomic or functional effects of desired genome engineering at selected sites to identify sites to be deselected due to off-target effects.

In some embodiments, provided is a system, comprising: at least one computer hardware processor; at least one database that stores a plurality of putative genomic target sites and/or a specified version of a genome reference sequence; and at least one non-transitory computer-readable storage medium storing processor-executable instructions that, when executed by the at least one computer hardware processor, cause the at least one computer hardware processor to perform: (a) seeding a search matrix with putative genomic target site nucleotide sequences having defined target specificity and degeneracy appropriate for the desired genome engineering application; (b) accessing and/or searching, in the at least one database, a specified version of a genome reference sequence to identify sites that share at least 95% identity with potential target sites defined in step (a). This identity refers to identity at the individual base pair level, with no gaps or additions with respect to the query sequence. Length variation is avoided by either excluding or disfavoring insertion or deletion variants. The search matrix can be generated from a source file of putative target sites, or an equivalent generated through an algorithm, based on target specificity defined at the DNA base pair level. Between the list of putative target sites and the reference sequence, one is searched against the other for hits at a pre-defined level of identity/homology.

The processor-executable instructions further cause the at least one computer hardware processor to perform: (c) selecting sites identified in (b) for which satisfaction of the following predefined criteria can be determined:
(i) unique in the reference genome sequence (no more than 1 site per haploid genome);
(ii) not in copy number-variable region;
(iii) target site does not contain nucleotide sequence or other genomic variation that would impede successful targeting;
(iv) at least 25 kilobases (kb) from an unannotated transcript;
(v) at least 50 kb from a 5′ gene end;
(vi) at least 50 kb from an ultra-conserved genomic region, enhancer, or other noncoding regulatory region;
(vii) at least 50 kb from a replication origin;
(viii) at least 300 kb from any microRNA or other functionally annotated small RNA;
(ix) at least 300 kb from a cancer-related gene.

In some embodiments, the processor-executable instructions further cause the at least one computer hardware processor to perform: (d) ranking the putative genomic target sites selected in step (c) according to the desired genome engineering application; and, optionally, assessing genomic or functional effects of desired genome engineering at selected sites to identify sites to be deselected due to off-target effects. In some embodiments, the ranking is based on the number of criteria (i)-(ix) that have been satisfied. In some embodiments, the ranking is based on a weighted scoring of criteria (i)-(ix). Weighted scoring can be used to tailor the results for suitability for the intended objective.

In some embodiments, the computer-implemented method is performed using the UCSC Genome Browser. Using this resource, one can activate tracks using the available menu features to load the sequence to be searched and to identify relevant criteria. For example, the selecting of step (c), in some embodiments, comprises receiving instructions to identify copy number variable regions [activate “Segmental Dups”], to identify all microRNAs [search “Sno/miRNA” in genome browser], to identify ultra-conserved regions [activate “GeneHancer”], identify replication origins and non-coding regulatory elements [activate “RefSeq Func Elems”], to identify all annotated transcripts and unannotated transcripts [activate “GENCODEv32”], and to identify regions of open chromatin [activate “ENCODE regulation”].

Example Embodiments

The following are exemplary embodiments of the materials and methods described herein.

Embodiment 1: A method of selecting genomic target sites for a desired genome engineering application, the method comprising: (a) seeding a search matrix with putative genomic target site nucleotide sequences having defined target specificity and degeneracy appropriate for the desired genome engineering application; (b) searching a specified version of a genome reference sequence to identify sites that share at least 95% identity with potential target sites defined in step (a); and (c) selecting sites identified in (b) for which satisfaction of the following predefined criteria can be determined: (i) unique in the reference genome sequence (no more than 1 site per haploid genome); (ii) not in copy number-variable region; (iii) target site does not contain nucleotide sequence or other genomic variation that would impede successful targeting; (iv) at least 25 kilobases (kb) from an unannotated transcript; (v) at least 50 kb from a 5′ gene end; (vi) at least 50 kb from an ultra-conserved genomic region, enhancer, or other noncoding regulatory region; (vii) at least 50 kb from a replication origin; (viii) at least 300 kb from any microRNA or other functionally annotated small RNA; (ix) at least 300 kb from a cancer-related gene.

Embodiment 2: The method of embodiment 1, further comprising: (d) ranking the putative genomic target sites selected in step (c) according to the desired genome engineering application; (e) validating target site presence in a targeted genomic sequence, cleavage efficiency of the site(s), and targeted insertion efficiency and fidelity of the transgene at the identified genomic target sites ranked in step (d); and, optionally, (f) assessing genomic or functional effects of desired genome engineering at selected sites to identify sites to be deselected due to off-target effects.

Embodiment 3: The method of embodiment 1, wherein the desired genome engineering application is transgene insertion, functional gene editing, cell marking, gene activation, or gene repression.

Embodiment 4: The method of embodiment 1, 2, or 3, wherein the search matrix comprises a position weight matrix (PWM).

Embodiment 5: The method of any of the preceding embodiments, wherein the selecting comprises selecting sites that satisfy each of the predefined criteria of (c).

Embodiment 6: The method of any of the preceding embodiments, wherein the ranking of step (d) assigns preference to criteria associated with safety, functional silence, and accessibility, respectively.

Embodiment 7: The method of any of embodiments 2-6, wherein the ranking of step (d) is based on searching genome browser data.

Embodiment 8: The method of embodiment 7, wherein the genome browser data are aggregated at and obtained from UCSC Genome Browser and/or Ensembl Genome Browser.

Embodiment 9: The method of any of embodiments 2-8, wherein the ranking of step (d) is based on scoring genomic target sites that satisfy the set of predetermined criteria of step (c).

Embodiment 10: The method of any of embodiments 2-9, wherein the ranking of step (d) is based on assessment of copy number variation and/or base pair level variation in sites identified in (b).

Embodiment 11: The method of embodiment 10, wherein the assessment comprises a survey of human population genomic variation data.

Embodiment 12: The method of any of embodiments 2-11, wherein the validating is performed in silica

Embodiment 13: The method of any of embodiments 2-12, wherein the validating for site presence and cleavage efficiency of step (d) comprises polymerase chain reaction (PCR) amplification of targeted sites and cleavage testing.

Embodiment 14: The method of any of embodiments 2-13, wherein the validating of step (e) comprises homology-dependent recombination (HDR) and/or non-homologous DNA end joining (NHEJ).

Embodiment 15: The method of any of embodiments 2-14, wherein the validating of step (e) comprises DNA sequencing, transgene expression and/or functional assays for a minimum of 10 cell population doublings to assess stability of transgene insertion and expression.

Embodiment 16: The method of any of embodiments 2-15, wherein the assessing of step (f) comprises genomic or functional assessments,

Embodiment 17: A method of ranking potential genomic target sites for desired genome engineering comprising performing the method of any of embodiments 2-16.

Embodiment 18: A method of producing a targeting construct for insertion of a transgene into a genomic site comprising: selecting a genomic targeting site according to a method described herein; and synthesizing a construct comprising the transgene flanked by application-specific 5′ and 3′ regulatory sequences, and target site-specific, transgene-flanking homology dependent sequences having sufficient nucleotide sequence homology or identity with the target site sequence to promote transgene insertion into the target site, or homology-independent repair sequence.

Embodiment 19: A targeting construct produced by the method of embodiment 18.

Embodiment 20: The targeting construct of embodiment 19, wherein the genomic targeting site of (a) is located on chromosome 2p (SHS229), chromosome 4q (SHS231), or on the short arm of chromosome 2, 5, or X, or on the long arm of chromosome 7, 14, or 17 (SHS253).

Embodiment 21: The targeting construct of embodiment 19, wherein the genomic targeting site of (a) has the cleavage specificity of the homodimeric I-Crel homing endonuclease and its monomerized derivative mCrel.

Embodiment 22: The targeting construct of embodiment 19, wherein the genomic targeting site of (a) is selected from the group consisting of the targeting sites listed in Table 2.

Embodiment 23: A system for selecting genomic target sites for a desired genome engineering application, the system comprising a user device comprising a hardware processor that is programmed to perform the method of any one of embodiments 1-17.

Embodiment 24: A non-transitory computer-readable medium containing computer executable instructions that, when executed by a processor, cause the processor to perform the method of any one of embodiments 1-17.

### EXAMPLES

The following examples are presented to illustrate the present invention and to assist one of ordinary skill in making and using the same. The examples are not intended in any way to otherwise limit the scope of the invention.

### Example 1

### New Human Chromosomal Sites with “Safe Harbor” Potential for Targeted Transgene Insertion

This Example reports the identification of 35 potential new human SHS, located on 16 different human chromosomes and 23 chromosome arms including both arms of the human X chromosome. These 35 new SHS and the three canonical human SHS (AAVS1, the human

ROSA26 locus and CCR5) were assessed and rank-ordered for safety and potential utility using a comprehensive scoring system that included 8 different genomic criteria in addition to uniqueness. Several high-ranking potential new SHS were experimentally validated by PCR amplification, mCrel cleavage sensitivity and DNA sequencing, together with a demonstration of efficient editing and transgene insertion mediated by Cas9, TALEN and mCrel nucleases. SHS-specific transgene insertion by both homology-mediated as well as cleavage-dependent, likely homology-independent mechanisms was demonstrated. The most extensively characterized of these new SHS, the high-ranking SHS231 located on the proximal long arm of chromosome 4, was also shown to be functionally competent for recombinase/integrase-mediated editing. Selectable, scorable and fluorescent/functional protein-encoding SHS231 transgenes were shown to be stably expressed when compared with the same transgenes inserted into the canonical AAVS1 site in a number of different human cell lines. The SHS231 engineering toolkit will allow others to make rapid use of this enhanced chromosome 4 SHS for both basic and clinically-oriented genome engineering applications.

Materials and Methods

Cell Lines/Cell Culture

Human 293T cells or derivatives and four human rhabdomyosarcoma (RMS) cell lines derived from unrelated patients were used for experiments. All five lines were cultured in D-MEM medium supplemented with 10% (v/v) fetal bovine serum (Hyclone, GE Healthcare/Biosciences, Pittsburgh, Pa.), 2 mM L-glutamine and antibiotics (1% Pen-Strep, Gibco, Thermo Fisher Scientific, Waltham, Mass.) in a 5% CO2 humidified 37° C. incubator. Human 293T-REX cells, a derivative of the parent 293T cell line (ATCC cell line CRL-3216), were grown in accordance with the supplier's instructions (Invitrogen/Thermo Fisher, Waltham, Mass.). The human RMS cancer cell lines RD, Rh5, Rh30 and SMSCTR have been described previously (10), and were obtained the laboratories of Dr. Corinne Linardic (Duke University School of Medicine, Durham, N.C.) and Dr. Charles Keller (Children's Cancer Therapy Development Institute, Beaverton, Oreg.). Cells were tested periodically for Mycoplasma infection and authentication was done by DNA fingerprinting (the RMS lines were verified by the Dana Farber Cancer Institute Molecular Diagnostic Laboratory by short tandem repeat profiling).

SHS identification and experimental validation

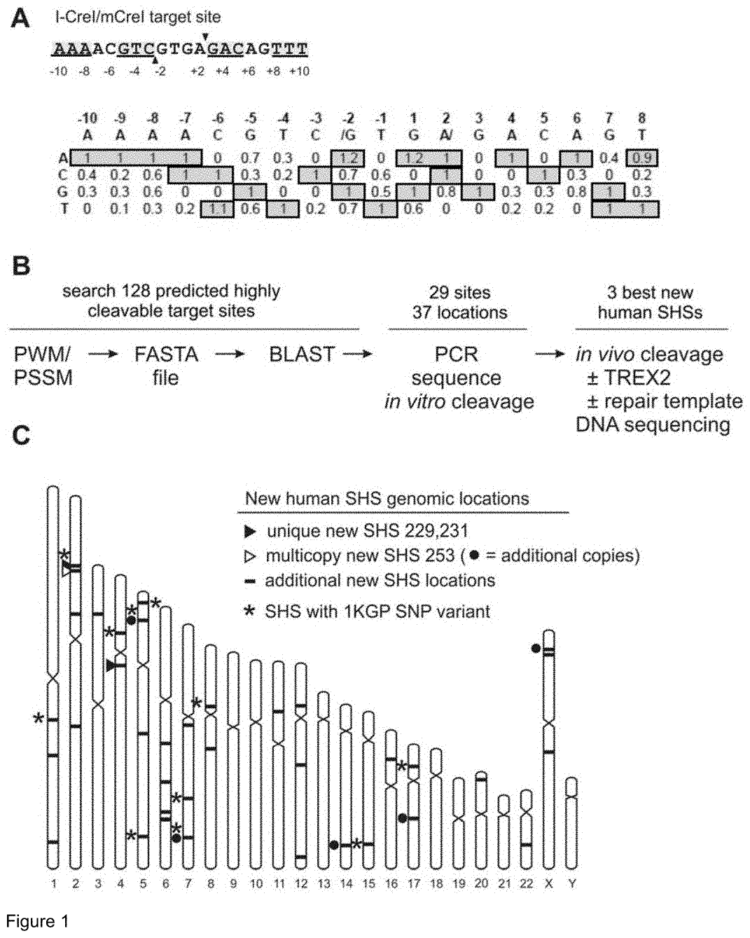

In order to identify potential new human SHS, we first searched the human genorne for high quality matches to the target sequence of the canonical homing endonuclease mCrel. We reasoned that a SHS identified by a highly cleavage-sensitive mCrel target site or variant would also contain one or more adjacent cleavage sites for Cas9 and TALEN-based nucleases that have less stringent targeting requirements. The well-defined mCrel site would also anchor the search of adjacent chromosomal DNA to assess and rank-order SHS suitability based on criteria for site safety, functional competence and the presence of potentially confounding sequence variations. This search was initiated by using detailed information on the cleavage specificity of rnCrel that quantified the contribution of each basepair in the rnCrel target site sequence. This position weight matrix was used to construct a list of 128 target site sequence variants predicted to be cleaved with ≥90% of the efficiency of the native mCrel site (11-16) (FIGS. 1A and 1B). These 128 mCrel target site variants were FASTA-formatted and uploaded to the NCBI BLAST search engine (http://blast.ncbi.nlm.nih.gov/) in order to identify target site matches in the human genome (GRCh37/hg19) using the following BLAST parameters: optimize for ‘Highly similar sequences (megablast)’; max target reqs=50; short queries: ‘adjust for short sequences’: expect threshold=1; word size=7; match/mismatch: 4, −5; and gap cost: existence=12/extension=8. All resulting genomic target site matches of ≥95% identity (19/20 or 20/20 bp matches versus the canonical mCrel target site) were subsequently evaluated as potential new safe harbor sites.

Potential new human SHS identified by BLAST search and the canonical human SHS AAVS1, HsROSA26 and CCR5 were then evaluated for SHS potential by 8 criteria in addition to site uniqueness that assessed site safety, accessibility and functional criteria (FIG. 1C; Tables 1 and 2). These criteria were based on several less extensive lists of criteria (e.g., proximity to known genes or regulatory elements, see, e.g., Sadelain et al 2012 (17)), and made use of contemporary genomic data, e.g., ENCODE Consortium project results (18). All SHS candidates including the three canonical human SHS were evaluated as follows: sites were first searched 300 kb up-and downstream in the UCSC Genome Browser in order to identify genes or RNAs, especially any already related to cancer; proximity to any transcriptionally active region regardless of annotation; the presence of replication origins or ultra-conserved elements; location in open chromatin as assessed by nuclease sensitivity; and whether the SHS was located in a region of copy number variation (19, 20) (CNV; genome.ucsc.edu/). We next used 1000 Genomes Project (1KGP) data (ncbi.nlm.nih.gov/variation/tools/1000genomes/) to identify basepair-level population genetic variation within all of the mCrel-anchored SHS sites (21) (Table 4). This approach was used to provide an estimate of the fraction of SHS that would be directly accessible in individuals by mCrel (and, by extension, other genome engineering nucleases). New SHS that differed from the canonical mCrel site at 1 or more basepair positions were further assessed using the mCrel position weight matrix (PWM) developed from single base-pair profiling experiments (14,16) (FIG. 1B) to predict cleavage sensitivity.

TABLE 1

| | SHS criterion | UCSC browser track source |
|---|---|---|
| safety | 1. >300 kb from any cancer- | genes and gene predictions: |
| | related gene on allOnco list | UCSC Genes |
| | 2. >300 kb from any miRNA/ | genes and gene predictions: |
| | other functional small RNA | sno/miRNA |
| | 3. >50 kb from any | genes and gene predictions: |
| | 5′ gene end | RefSeq Genes |
| functional | 4. >50 kb away from | regulation: UW Repli-seq: |
| silence | any replication origin | Peaks |
| | 5. >50 kb away from any | regulation: |
| | ultraconserved element | VISTA Enhancers |
| | 6. low transcriptional | mRNA and EST: |
| | activity (no mRNA ± 25 kb) | Human mRNAs |
| consistent/ | 7. not in copy number | repeats: Segmental Dups |
| accessible/ | variable region | |
| unique | 8. in open chromatin | regulation: ENC DNase/ |
| | (DHS signal ± 1 kb) | FAIRE: Uniform DNasel HS |
| | unique | BLAST search output |
| | (1 copy in human genome) | |

TABLE 2
Criteria for identfication and assessment of new human safe harbor sites

| | | | | |
|---|---|---|---|---|
| | | SEQ ID | Site | |
| Genomic location | Sequence | NO | score | Site ID |

Current human SHSs

| | | | | |
|---|---|---|---|---|
| chr19: 55,625,241-55,629,351 | | | 5 | AAVS1 |
| chr3: 46,414,443-46,414,942 | | | 3 | CCR5 |
| chr3: 9,415,082-9,414,043 | | | 3 | hROSA26 |
| Canonical I-CreI/mCreI site | AAAACGTCGTGAGACAG | 51 | | |

New human SHSs

| | | | | |
|---|---|---|---|---|
| chr1: 152,360,840-152,360,859 | AAAATGTCAgGAGACATTTT | 1 | 4 | 323 |
| chr8: 68,720,172-68,720,191 | ″ | 1 | 7 | 325 |
| chr1: 175,942,362-175,942,381 | AAACTGTCATGAGACATTTg | 2 | 2 | 289 |
| chr1: 231,999,396-231,999,415 | AAACTGTCATGgGACAGATT | 3 | 5 | 227 |
| *chr2: 45,708,354-45,708,373 | AAAATGTCATGCGACATTTT | 4 | 5 | 229 |
| *chr2: 48,830,185-48,830,204 | AAACTGaCATAAGACAGATT | 5 | 4 | 253 |
| chr5: 19,069,307-19,069,326 | ″ | 5 | 5 | 255 |
| chr7: 138,809,594-138,809,613 | ″ | 5 | 4 | 257 |
| chr14: 92,099,558-92,099,577 | ″ | 5 | 5 | 259 |
| chr17: 48,573,577-48,573,596 | ″ | 5 | 4 | 261 |
| chrX: 12,590,812-12,590,831 | ″ | 5 | 5 | 263 |
| chr2: 77,263,930-77,263,949 | AAAATGTgGTGAGACATTTT | 6 | 6 | 317 |
| chr2: 150,500,675-150,500,694 | AAACTGTCATAAGACAGATc | 7 | 7 | 303 |
| chr3: 31,670,871-31,670,890 | AAAATGTCATACtACAGATT | 8 | 5 | 331 |
| chr4: 37,769,238-37,769,257 | AAACCGTCGTGAtACATTTT | 9 | 6 | 283 |
| *chr4: 58,976,613-58,976,632 | AAACTGTCATAtGACAGATT | 10 | 7 | 231 |
| chr5: 7,577,728-7,577,747 | AAAATGTCATGAGACAGTcT | 11 | 5 | 315 |
| chr5: 93,159,222-93,159,241 | AAAATGTCAaGAGACATTTT | 12 | 3 | 327 |
| chr5: 159,922,029-159,922,048 | AAACTGTCAaAAGACAGATT | 13 | 3 | 305 |
| chr16: 19,323,777-19,323,796 | ″ | 13 | 5 | 307 |
| chr20: 5,055,245-5,055,264 | ″ | 13 | 4 | 309 |
| chr6: 89,574,320-89,574,339 | AAACTGTCcTAAGACAGTTT | 14 | 5 | 285 |
| chr6: 114,713,905-114,713,924 | AAAATtTCATGAGACATTTT | 15 | 7 | 233 |
| chr6: 134,385,946-134,385,965 | AAAATGTCATGAGgCAGTTT | 16 | 6 | 311 |
| chr6: 138,972,461-138,972,480 | AAACTGTCATACcACAGTTT | 17 | 4 | 299 |
| chr7: 113,327,685-113,327,704 | AAACTGTCATACaACAGTTT | 18 | 6 | 301 |
| chr8: 40,727,927-40,727,946 | AAACTGaCGTAAGACAGATT | 19 | 6 | 293 |
| chr11: 32,680,546-32,680,565 | AAAATGTCcTGAGACAGATT | 20 | 5 | 319 |
| chr12: 27,543,737-27,543,756 | AAAAaGTCATGAGACATTTT | 21 | 4 | 333 |
| chr12: 66,516,386-66,516,405 | AAACTGTaGTAAGACAGATT | 22 | 4 | 295 |
| chr12: 126,152,581-126,152,600 | AAAATGTCATGAGAtATTTT | 23 | 5 | 329 |
| chr17: 14,810,285-14,810,304 | AAACaGTCATAAGACAGATT | 24 | 4 | 297 |
| chr22: 35,770,121-35,770,140 | AAACTGaCATGAGACAGATT | 25 | 4 | 291 |
| chrX: 16,059,732-16,059,751 | AAAATGTCATGAGAaAGTTT | 26 | 6 | 313 |
| chrX: 79,674,328-79,674,347 | AAAATGTCATAAGgCAGTTT | 27 | 3 | 321 |

| Cre site | Table 1 site criterion | | | | | | | | Site | |
|---|---|---|---|---|---|---|---|---|---|---|
| match | 1 | 2 | 3 | 4 | 5 | 6 | 7 | 8 | score | Site ID |
| | − | + | − | + | + | − | + | + | 5 | AAVS1 |
| | − | + | − | + | + | − | + | + | 5 | CCR5 |
| | − | + | − | − | + | − | + | − | 3 | hROSA26 |
| 19 | + | + | − | − | + | − | + | − | 4 | 323 |
| 19 | + | + | + | + | + | + | + | − | 7 | 325 |
| 19 | − | − | − | − | + | − | + | − | 2 | 289 |
| 19 | + | + | − | + | + | − | + | − | 5 | 227 |
| 20 | + | + | − | + | + | − | + | − | 5 | 229 |
| 19 | − | + | − | + | + | − | + | − | 4 | 253 |
| 19 | + | + | − | + | + | − | + | − | 5 | 255 |
| 19 | − | + | − | − | + | − | + | + | 4 | 257 |
| 19 | + | + | − | + | + | − | + | − | 5 | 259 |
| 19 | − | + | − | + | + | − | + | − | 4 | 261 |
| 19 | + | + | − | + | + | − | + | − | 5 | 263 |
| 19 | + | + | − | + | + | − | + | + | 6 | 317 |
| 19 | + | + | + | + | + | + | + | − | 7 | 303 |
| 19 | + | + | − | + | + | − | + | − | 5 | 331 |
| 19 | + | + | − | + | + | − | + | + | 6 | 283 |
| 19 | + | + | + | + | + | + | + | − | 7 | 231 |
| 19 | + | + | − | + | + | − | + | − | 5 | 315 |
| 19 | − | − | − | + | + | − | + | − | 3 | 327 |
| 19 | − | − | − | + | + | − | + | − | 3 | 305 |
| 19 | + | + | − | + | + | − | + | − | 5 | 307 |
| 19 | − | + | − | − | + | − | + | + | 4 | 309 |
| 19 | + | + | − | + | + | − | + | − | 5 | 285 |
| 19 | + | + | + | + | + | + | + | − | 7 | 233 |
| 19 | + | + | − | + | + | − | + | + | 6 | 311 |
| 19 | + | − | − | + | + | − | + | − | 4 | 299 |
| 19 | + | − | + | + | + | + | + | − | 6 | 301 |
| 19 | + | + | − | + | + | − | + | − | 6 | 293 |
| 19 | − | + | − | + | + | − | + | + | 5 | 319 |
| 19 | − | + | − | + | + | − | + | − | 4 | 333 |
| 19 | − | + | − | + | + | − | + | − | 4 | 295 |
| 19 | + | + | − | + | + | − | + | − | 5 | 329 |
| 19 | + | − | − | + | + | − | + | − | 4 | 297 |
| 19 | − | + | − | + | + | − | + | − | 4 | 291 |
| 19 | − | + | + | + | + | + | + | − | 6 | 313 |
| 19 | − | + | − | − | + | − | + | − | 3 | 321 |

Groups of sites that share the same mCreI target site sequence, but are found at different sites in the human genome, are indicated with ″; * identifies three newly identified SHS chosen for additional genomic and/or functional characterization.

Potential new SHS identified and assessed by the above criteria were then rank-ordered and experimentally validated by PCR amplification and mCrel in vitro cleavage analyses. Site-specific primer pairs were designed using CLC Workbench Primer Design Tool (clcbio.com; CLC Bio, Boston, Mass.) to generate ˜300-400 bp PCR products containing the mCrel target site (Table 3). Genomic DNA purified from human 293T cells using a Wizard Genornic DNA Purification Kit (Promega, Madison, Wis.) was used as the template for SHS amplifications (Table 3). SHS amplification reactions were performed in 25 μL of 1× Thermo polymerase buffer containing all four dNTPs at 200 μM, 150 ng of genomic DNA and 400 nM of each primer with 1.25 units of Taq polymerase (New England Biolabs; NEB, Ipswich, Mass.). Amplifications were performed using a 1 min 95° C. denaturation step followed by 30 cycles of 30 sec at 95° C.; 30 sec at 50° C.; and 30 sec at 68° C. followed by 5 min at 68° C. Alternatively, a subset of SHS was amplified in 25 μL reactions that contained 12.5 μL PrimeStar Max DNA polymerase premix (Takara, Mountain View, Calif.), 50 ng of purified genomic DNA and 240 nM final concentration for each amplification primer. Amplifications were performed using 35 cycles of 10 sec at 98° C.; 15 sec at 50° C. and 3 min at 72° C. SHS-specific PCR products were gel-purified using a QIAquick Gel Extraction Kit (Qiagen, Hilden, Germany), quantified by spectrophotometry, then digested with purified mCrel protein in 15 μL reactions containing 15 fmol DNA substrate and 0, 15 or 150 fmol of purified mCrel protein (8, 16) in 170 mM KCl, 10 mM MgCl2 and 20 mM Tris pH 9.0. Digestions were performed at 37° C. for 1 hr, then stopped by adding 3 μL (1:6) of 6× stop buffer (60 mM Tris, HCl pH 7.4, 3% SOS, 30% glycerol, 150 mM EDTA) prior to electrophoresis through a 1% agarose gel run in TAE buffer (40 mM Tris, 20 mM acetic acid, 1 mM EDTA). Substrate and cleavage product bands were identified following gel electrophoresis by ethidium bromide staining, digital image capture and band intensity quantification using ImageJ (http://imagej.nih.gov/ij/). A comparably-sized PCR product containing the native mCrel target site was included in experiments as a positive digestion control. A subset of newly identified SHS were also sequence-verified from PCR products using SHS-specific primers by capillary sequencing (Table 3; Genewiz, South Plainfield, N.J.). Sequenced reads were aligned to genomic sequence using CLC Workbench Alignment tool (CLC Bio, Boston, Mass.).

TABLE 3
Sequences of primers used for SHS amplification, sequencing, and vector
construction

| | Expected | | | | |
|---|---|---|---|---|---|
| | Amplicon | | | | SEQ |
| Site | Size | | | | ID |
| ID | (in bp) | Purpose | Polarity | Sequence (5′→3′) | NO: |
| 225 | | Sequencing | | CGAACGCCGGGTTAAGGC | 52 |
| | 3,053 | Amplifi- | Forward | CCTGCCGAATCAACTAGC | 53 |
| | | cation | | | |
| | | | Reverse | GACAAACCCTTGTGTCGA | 54 |
| 227 | | Sequencing | | GCGCCTGGCCTAAAACATTC | 55 |
| | 456 | Amplifica- | Forward | TTTAGTAGAGAAGGGGTTTC | 56 |
| | | tion | | | |
| | | | Reverse | CTTCTGATCTACACTGGTCC | 57 |
| | 4,910 | Amplifica- | Forward | GGACTGGTTATCTGTCTAAC | 58 |
| | | tion | | | |
| | | | Reverse | CTCAGAGGTCTGGACACA | 59 |
| 229 | | Sequencing | | GCTCAGATGATCATTAGCATT | 60 |
| | 478 | Amplifica- | Forward | TAAGAAACTGCCACCACATC | 61 |
| | | tion | | | |
| | | | Reverse | CCATAACTCTTCCTCTCTCT | 62 |
| | 1,134 | Amplifica- | Forward | GAAGATGCTATGAACGTTGTGG | 63 |
| | | tion | | | |
| | | | Reverse | GGCAAATAACATTCTATTGTATGGG | 64 |
| | 4,930 | Amplifica- | Forward | CCACAACAGTAAACCAAGTC | 65 |
| | | tion | | | |
| | | | Reverse | CCTGTCTGATGTCAAGGAGA | 66 |
| | 1,180 | Repair | Rt Fwd | GAAGATGCTATGAACGTTGTGG | 67 |
| | | template | Rt Rev | CCGCGGATAACTTCGTATAATGTATGCTATACG | 68 |
| | | construc- | | AAGTTATCGATCGGCAT | |
| | | tion | | | |
| | | | Lt Fwd | CGATCGATAACTTCGTATAGCATACATTATACG | 69 |
| | | | | AAGTTATCCGCGGATGC | |
| | | | Lt Rev | GGCAAATAACATTCTATTGTATGGG | 70 |
| 231 | | Sequencing | | GCATTCTTTAGTGGTTGTGAA | 71 |
| | 411 | Amplifica- | Forward | TATCTGGGAAAGGGTCATCT | 72 |
| | | tion | | | |
| | | | Reverse | CCCCTTGCCTTGTTCCATTT | 73 |
| | 1,020 | Amplifica- | Forward | GCTGCTCAGCTAAGCATAGC | 74 |
| | | tion | | | |
| | | | Reverse | GAAGGAGTTCAGAACACATTATCC | 75 |
| | 4,888 | Amplifica- | Forward | GTCACAAATTGCATTGCATT | 76 |
| | | tion | | | |
| | | | Reverse | CCTGCAACAATATTCTCACT | 77 |
| | 1,066 | Repair | Rt Fwd | GCTGCTCAGCTAAGCATAGC | 78 |
| | | template | Rt Rev | CCGCGGATAACTTCGTATAATGTATGCTATACG | 79 |
| | | construc- | | AAGTTATCGATCGATAT | |
| | | tion | | | |
| | | | Lt Fwd | CGATCGATAACTTCGTATAGCATACATTATACG | 80 |
| | | | | AAGTTATCCGCGGATAT | |
| | | | Lt Rev | GAAGGAGTTCAGAACACATTATCC | 81 |
| 233 | | Sequencing | | GGCTGAGGCAGGAGAATTGA | 82 |
| | 459 | Amplifica- | Forward | TTACCTGAGGTCAGGTAATC | 83 |
| | | tion | | | |
| | | | Reverse | GCCTGACTTGATCGTTCTAC | 84 |
| | 4,731 | Amplifica- | Forward | GGAGCCCTAATCCAATATGC | 85 |
| | | tion | | | |
| | | | Reverse | CCTTATGAATGTTTTAAATCTC | 86 |
| 235 | | Sequencing | | CCAGCCTGGGTGACAGAG | 87 |
| 237 | | Sequencing | | GGTTAAGTAAGGCCAAATTAATG | 88 |
| 251 | | Sequencing | | GCTGTTTTTGAGAATACCCTC | 89 |
| | 439 | Amplifica- | Forward | TTTGCATGGCTTCTTCCCTC | 90 |
| | | tion | | | |
| | | | Reverse | TTGGGAAAGTTGCTTATAGG | 91 |
| 253 | | Sequencing | | GTGTCACTGAAGTGAGAGCAA | 92 |
| | 439 | Amplifica- | Forward | GCTGCTAGAGTAAGATGAGG | 93 |
| | | tion | | | |
| | | | Reverse | CGTTAATTTCCCCCATGTAT | 94 |
| | 1,023 | Amplifica- | Forward | GGAGACAGCAAGTAGCAATTGAATG | 95 |
| | | tion | | | |
| | | | Reverse | GCCAAGCAAATGCTGGTTCC | 96 |
| | 4,944 | Amplifica- | Forward | GCTGTCAAATACAGTTTTACACA | 97 |
| | | tion | | | |
| | | | Reverse | CCCATTGGTAAGTAATGCATG | 98 |
| | 1,069 | Repair | Rt Fwd | GGAGACAGCAAGTAGCAATTGAATG | 99 |
| | | template | Rt Rev | CCGCGGATAACTTCGTATAATGTATGCTATACGAAG | 100 |
| | | construc- | | TTATCGATCGTTA | |
| | | tion | | | |
| | | | Lt Fwd | CGATCGATAACTTCGTATAGCATACATTATACGAAG | 101 |
| | | | | TTATCCGCGGATAA | |
| | | | Lt Rev | GCTGTCAAATACAGTTTTACACA | 102 |
| 255 | | Sequencing | | GACACCTTCTATTATATTTCGAT | 103 |
| | 441 | Amplifica- | Forward | CACCAGTTGAAGTAAGACCT | 104 |
| | | tion | | | |
| | | | Reverse | CAGTGGCATGATCTGGAGTG | 105 |
| | 4,948 | Amplifica- | Forward | CTTCTGTGATGCCTTGAATC | 106 |
| | | tion | | | |
| | | | Reverse | GAGAACAAAATCCAAGCTTACT | 107 |
| 257 | | Sequencing | | GCCTCTATTCCCTTCTGTACC | 108 |
| | 404 | Amplifica- | Forward | TGTTCACCATACACTTCCTC | 109 |
| | | tion | | | |
| | | | Reverse | CAGATAAGCACAAATTCACC | 110 |
| | 4,995 | Amplifica- | Forward | GGTAAACTATACATCGGTTGGG | 111 |
| | | tion | | | |
| | | | Reverse | CCAAAACCTGGGTCACCAA | 112 |
| 259 | | Sequencing | | GGCCTAGGACTAGGCCATTC | 113 |
| | 409 | Amplifica- | Forward | GGAAGAGTTTAAGACTGGAA | 114 |
| | | tion | | | |
| | | | Reverse | ACCCTTATCTTCCTAGCCAC | 115 |
| | 4,984 | Amplifica- | Forward | GCTTACAGTAAGAGTCAATAACC | 116 |
| | | tion | | | |
| | | | Reverse | GCAATCAGAGTGATCCTTTC | 117 |
| 261 | | Sequencing | | CCACCGCGCCTAGCTGAG | 118 |
| | 478 | Amplifica- | Forward | TTTTTTTAGTAGAGACGGGG | 119 |
| | | tion | | | |
| | | | Reverse | TGGTAGATGTGGGGTTTCAC | 120 |
| | 4,937 | Amplifica- | Forward | GGATTAAGCAGTGAATGGG | 121 |
| | | tion | | | |
| | | | Reverse | CCACCATGTATATCCTTCCC | 122 |
| 263 | | Sequencing | | GGTGTCTATCTTATGCACTGT | 123 |
| | 363 | Amplifica- | Forward | GATGCTTTTTGTTATGGGGG | 124 |
| | | tion | | | |
| | | | Reverse | AGACAAGCTTCATTCACCAC | 125 |
| | 4,931 | Amplifica- | Forward | GAACTCCACTCTCTGAACT | 126 |
| | | tion | | | |
| | | | Reverse | ATGATGTTCAGGATAAAGTACACT | 127 |
| 283 | 469 | Amplifica- | Forward | GGCACCATTTTCTCATTAGC | 128 |
| | | tion | | | |
| | | | Reverse | TGGTTTTGTTGTGGGAGTCC | 129 |
| 285 | 391 | Amplifica- | Forward | TAACATATAGCAAAGAGGGG | 130 |
| | | tion | | | |
| | | | Reverse | TGCCCTCAAGTTTCATATGC | 131 |
| 287 | 401 | Amplifica- | Forward | GCTTTCTTTCCTCTGGGCAC | 132 |
| | | tion | | | |
| | | | Reverse | CCATTTATTGCTTGCTTTCC | 133 |
| 289 | 433 | Amplifica- | Forward | TTCAGTAGAGATGGGGTTTC | 134 |
| | | tion | | | |
| | | | Reverse | TACTGTGTTATGCTGACTTC | 135 |
| 291 | 399 | Amplifica- | Forward | GCTCTTCCTAGTCTCTTCTC | 136 |
| | | tion | | | |
| | | | Reverse | CCACCATGCCTATCTACCCC | 137 |
| 293 | 465 | Amplifica- | Forward | TCCAGACAACTTTTATTCCC | 138 |
| | | tion | | | |
| | | | Reverse | ATAGGACACGTAAGGAAAGA | 139 |
| 295 | 397 | Amplifica- | Forward | TTCAATCTGTCCCAAGCATC | 140 |
| | | tion | | | |
| | | | Reverse | AGTGTGTTCTTCAGTATCAG | 141 |
| 297 | 305 | Amplifica- | Forward | TGAGAGATGTATGTGAGGAC | 142 |
| | | tion | | | |
| | | | Reverse | TTCTTCCATGTCACTATCTG | 143 |
| 299 | 451 | Amplifica- | Forward | TAATAGCTACACATGCCAAC | 144 |
| | | tion | | | |
| | | | Reverse | AAAGAGGAGACAAGGTTAGG | 145 |
| 301 | 468 | Amplifica- | Forward | AAGGAACAGACCATGAGAAG | 146 |
| | | tion | | | |
| | | | Reverse | GGCTGCATCACTACATTATT | 147 |
| 303 | 401 | Amplifica- | Forward | CTACATGTTCTTTCTTCCCT | 148 |
| | | tion | | | |
| | | | Reverse | CCTCACTCCTCACATGTTCA | 149 |
| 305 | 377 | Amplifica- | Forward | TAAACCCCAAACCCCCTTTC | 150 |
| | | tion | | | |
| | | | Reverse | ACAGGAATGAGAGTAAGAAAG | 151 |
| 307 | 392 | Amplifica- | Forward | GAGGTTGAGGCTACAGTGAG | 152 |
| | | tion | | | |
| | | | Reverse | CCTCTAGAAAGCCAACCCTC | 153 |
| 309 | 345 | Amplifica- | Forward | TTCCCACAGTTTACAACCC | 154 |
| | | tion | | | |
| | | | Reverse | GATCTCACTATGTTGCCCA | 155 |
| 311 | 396 | Amplifica- | Forward | GTTTTGTGCTGACATTGGAG | 156 |
| | | tion | | | |
| | | | Reverse | CTACCACTTTACTTCTCATCAG | 157 |
| 313 | 447 | Amplifica- | Forward | CACGTTAAAAAACAAAAGAC | 158 |
| | | tion | | | |
| | | | Reverse | GAGGAATGCAGAATGTTAGC | 159 |
| 315 | 359 | Amplifica- | Forward | AAAAGGCAATGGTGTGTATG | 160 |
| | | tion | | | |
| | | | Reverse | CATTTTTCTTTTCGCTGGTC | 161 |
| 317 | 419 | Amplifica- | Forward | CTGTGGAATATTGATGCTAT | 162 |
| | | tion | | | |
| | | | Reverse | TTTGAGGGGACAGCTAGGGA | 163 |
| 319 | 362 | Amplifica- | Forward | GTGACTAAGTGAAACTGGAA | 164 |
| | | tion | | | |
| | | | Reverse | CATGCAACTCTCCTTTCAAA | 165 |
| 321 | 464 | Amplifica- | Forward | CCTCCTATCTTCTTTCTCAC | 166 |
| | | tion | | | |
| | | | Reverse | GTGAAGAATAGAGGTAGGGT | 167 |
| 323 | 405 | Amplifica- | Forward | GCCAACCTCATTCTACTTTT | 168 |
| | | tion | | | |
| | | | Reverse | GAATTAGAGGATAGGCAGCA | 169 |
| 325 | 352 | Amplifica- | Forward | CAGAGGTGATAACAGATACA | 170 |
| | | tion | | | |
| | | | Reverse | GTTCCTGATTGTGTTGGTTT | 171 |
| 327 | 374 | Amplifica- | Forward | ACACATAATCTTAACTCCAAG | 172 |
| | | tion | | | |
| | | | Reverse | GGTGACAGAGCTTTTTAGTG | 173 |
| 329 | 431 | Amplifica- | Forward | TCTTTGTAGTTGCTGTTTGC | 174 |
| | | tion | | | |
| | | | Reverse | GGAAAAGGGGGTTGATATAG | 175 |
| 331 | 306 | Amplifica- | Forward | GGGAAATGAAAAGAGGAAAC | 176 |
| | | tion | | | |
| | | | Reverse | GCACATTTCTCTTCAGCACA | 177 |
| 333 | 347 | Amplifica- | Forward | CTTAAGATGTTCCAGGTGTG | 178 |
| | | tion | | | |
| | | | Reverse | TTACCGTTTCAGGTGTTTGT | 179 |
| 335 | 348 | Amplifica- | Forward | GGCCTGCTTCTCCTCAGCTT | 180 |
| | | tion | | | |
| | | | Reverse | GTGACGTAAAGCCGAACCCG | 181 |
| 337 | 370 | Amplifica- | Forward | CTAAGGGAACAAATGGTGAA | 182 |
| | | tion | | | |
| | | | Reverse | TGAGTGGGTTTACTTGAGTG | 183 |

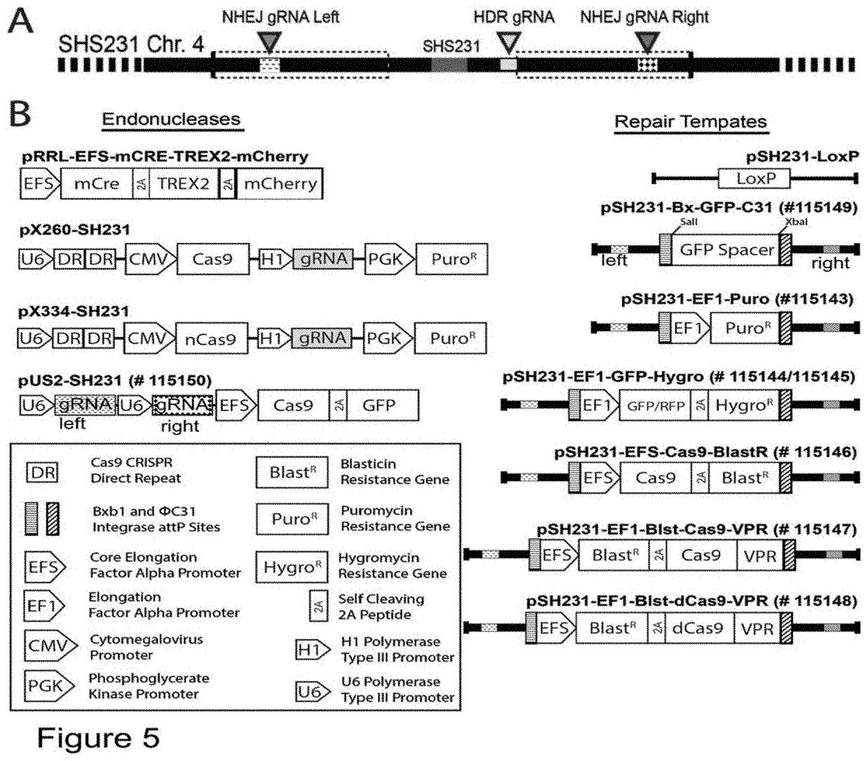

We verified the in vivo cleavage sensitivity of several potential SHS by co-expressing the mCrel homing endonuclease together with the TREX2 3′ to 5′ repair exonuclease in 293T cells. The inclusion of TREX2 allows a more accurate measure of the fraction of sites cleaved in vivo by promoting NHEJ-mediated mutagenic repair following site cleavage (22) (FIG. 5). The expression vector used in these experiments was constructed in a pRRL-based lentiviral vector backbone that encoded the open reading frames for mCrel, the TREX2 exonuclease and mCherry fluorescent protein in a single translational unit separated by self-cleaving T2A peptides (25) (FIG. 5). Target site cleavage was estimated by amplifying sites from transfected cells, then determining the fraction of PCR products that were mCrel cleavage-resistant and mutant. We extensively analyzed three new SHS in this way: SHS231, a unique chromosome 4 site with the highest SHS score; SHS229, a chromosome 2 SHS with perfect nucleotide sequence identity to a member of our 20 bp site query library; and SHS253, the chromosome 2-specific member of the small family of 6 identical target sites represented once each on 6 different chromosomes (chromosomes 2, 5, 7,14,17 and X; FIG. 1C, Table 2).

A modified calcium phosphate (CaPO4) transfection protocol (23) was used to introduce a pRRL-based lentiviral expression vector encoding mCrel, TREX2 and mCherry proteins into human 293T cells (24) (FIG. 5). Cells (2-4×10e5/well) were plated in a 6-well plate 24 hr prior to transfection and were ˜70% confluent at the time of transfection. Expression vector plasmid DNA (1.5 μg in 10 μL H2O) was mixed with 40 μL of freshly prepared 0.25 M CaCl2 and 40 μL of 2× BBS buffer (50 mM BES pH 6.95 (NaOH), 280 mM NaCl, 1.5 mM Na2HPO4; Boston BioProducts), then incubated at room temperature for 15 min before being added dropwise to wells. Plates were incubated overnight in 3% CO2 at 37° C. The medium was changed the following day, and cells were grown for an additional 24 hr in a 5% CO2, 37° C. humidified incubator. Transfection efficiency was checked by determining the fraction of mCherry-positive cells by flow cytometry: in brief, cells were trypsinized, counted and fixed with formaldehyde (1% v/v final concentration, 10 min at room temperature followed by the addition of 1/20 volume of 2.5 M glycine) prior to flow cytometric analysis of ˜2×10e4 cells/transfection on a BD FACS Canto II flow cytometer (BD Biosciences, San Jose, Calif.). Genomic DNA prepared from co-transfected and control cells was used for PCR amplification and in vitro mCrel cleavage analysis of specific SHS as described above.

Homology-Dependent SHS Editing by Three Genome Engineering Nucleases

The mCrel-I expression vector described above, together with SHS231-specific TALEN and CRISPR/Cas9 expression vectors, were used for SHS editing experiments. The SHS231-specific TALEN protein pair was designed using the TALEN Targeter 2.0 web design engine (26,27) (https://tale-nt.cac.cornell.edu/node/add/talen), Forward and reverse strand, 20 bp-specific TALEN sequences were inserted into the TALEN expression vector pRKSXX-pCVL-UCOE.7-SFFV-BFP-2A-HA-NLS2.0-TruncTAL (Dr. Andrew Scharenberg, Seattle Children's Research Institute, Seattle Wash.), and each TALEN open reading frame was generated by assembling the following repeat variable di-residues (RVDs): left TALEN: NG NG NN NN HD NG NI NH NN NH HD NG NI NI NN NN NI NG NG NI, corresponding to the nucleotide sequence TTGGCTAGGGCTAAGGATTA (SEQ ID NO: 30; chr 4: 58,976,594-58,976,613); and right TALEN: NG NN NG NI NG NH HD NG NG NG HD HD NG HD NG NG NN NG NG NI, corresponding to the nucleotide sequence TGTATGCTTTCCTCTTGTTA (SEQ ID NO: 31) (26,28) (chr 4:58,976,613-58,976,632),

A SHS231-specific CRISPR/Cas9 expression vector was constructed in pX260 (29,30) that contained expression cassettes for the S. pyogenes Cas9 nuclease, the CRISPR RNA array, and the tracrRNA. The SHS231 Cas9 target site, 5′-AAAACATTTATATACTGCGTGG-3′ (SEQ ID NO: 32), was located 110 bp downstream of the mCrel/TALEN cleavage site, was identified using the CRISPR Design Tools Resource developed by Zhang and colleagues (29,30) (crispr,mit.edu/). A corresponding SHS231-specific Cas9 nickase expression vector was also constructed in pX334, which encoded a Cas9 D10A substitution to confer nickase activity. A guide RNA template sequence, 5′-CTAATCTGGACAAAACATTTATATACTGCG-3′ (SEQ ID NO: 33), was inserted into both expression vectors followed by a TGG proto-spacer adjacent (PAM) motif (29,30).

In order to determine whether SHS cleavage in vivo could catalyze homology-directed repair in the presence of a homologous donor template, we co-transfected human 293T cells with a SHS-specific repair template and an expression vector for mCrel, for a TALEN pair, or for Cas9 cleavage/nickase enzymes (FIG. 2, FIG. 5). The template for SHS-specific, homology-dependent repair consisted of 500 bp homology arms that flanked the mCrel target site region and contained a 48 bp insert at the center harboring a canonical loxP recombinase site and adjacent, diagnostic restriction endonuclease cleavage sites for Pvul and SaclI (FIG. 2). Repair templates were made by overlap extension PCR using oligonucleotide primers to generate PCR products that, when re-amplified, incorporated the 48 bp loxP insert at the center of the repair template (Table 3).

Calcium phosphate transfection (as described above) was again used to introduce nuclease expression vectors into human 293T cells (24). Transfection efficiency was checked by determining the fraction of mCherry-positive cells by flow cytometry, as described above.

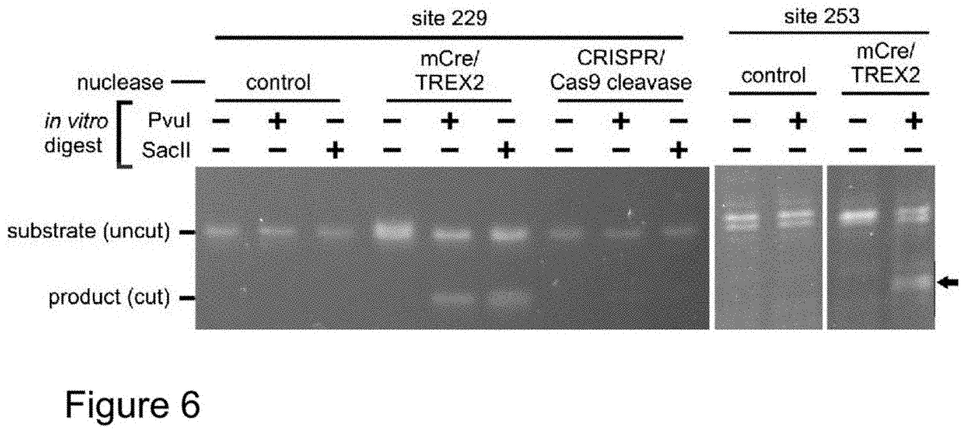

Molecular characterization of SHS editing was performed by PCR amplifying the SHS region of interest from transfected cells, followed by Pvul or SaclI restriction digest to confirm targeted integration of the loxP cassette (FIG. 2, FIG. 6). PCR products were also cloned into a pGEM-T Easy plasmid vector (Promega, Madison, Wis.) and transformed into α-Select Chemically Competent Gold Efficiency cells (Bioline, Taunton, Mass.), followed by plasmid preparation from white (insert-containing) colonies for capillary sequencing using a T7 promoter sequencing primer (FIG. 2). Sequencing results were aligned with the repair template sequence using the CLC Main Workbench software (CLCBio).

Homology-Independent SHS Genome Editing by Cas9

Homology-independent editing of the SHS231 locus was performed using the protocol above with modified Cas9 and repair template constructs. Dual human US-driven guide RNAs (gRNA) targeting SHS231 were simultaneously inserted into a custom S. pyogenes Cas9-T2A-GFP expression plasmid (pUS2-SH231) using Gibson assembly, as previously described 31. SHS231-specific gRNAs (SHS231 gRNA1: 5′-GCCTCCCCCATAGTACCAT-3′ (SEQ ID NO: 34); SH231 gRNA2: 5′-GATGTGCTCACTGAGTCTGA-3′ (SEQ ID NO: 35)) were designed to target and cleave both the SHS231 genomic locus and the repair template to promote efficient transgene integration by NHEJ-mediated DNA end joining (32,33). The transgene cassettes were also flanked by Bxb1 recombinase and ϕC31 attP integrase target sites that, once integrated, could be used for high efficiency SHS-specific editing by these recombinase/integrase proteins.

To engineer SHS231 using homology-independent approaches, repair templates (3 μg) and the pUS2-SH231 dual guide-targeting Cas9 expression vector (3 μg) were co-electroporated into three different human rhabdomyosarcoma (RMS) cell lines (Rh5, Rh30, and SMSCTR10; 1×10e6 cells per transfection) using the 100u1 Neon electroporation system (Life Technologies, Carlsbad, Calif.) according to the manufacturer's protocol and two, 1150V pulses for 30 ms each. After 2 weeks of selection (puromycin, hygromycin or blasticin, depending on the repair template; see FIG. 1, Table 5), transgene integration was confirmed with PCR amplification of the SHS231 target site (Q5 polymerase, NEB, Ipswich, Mass.) using a transgene and adjacent genome-anchored primer pair (SHS231 gFwd: GAACCAGAGCCACCCAGTTG (SEQ ID NO: 36), and Bxb1 rev; GTTTGTACCGTACACCACTGAGAC (SEQ ID NO: 37)).

Stable Gene Expression from SHS231 Transgene Insertions

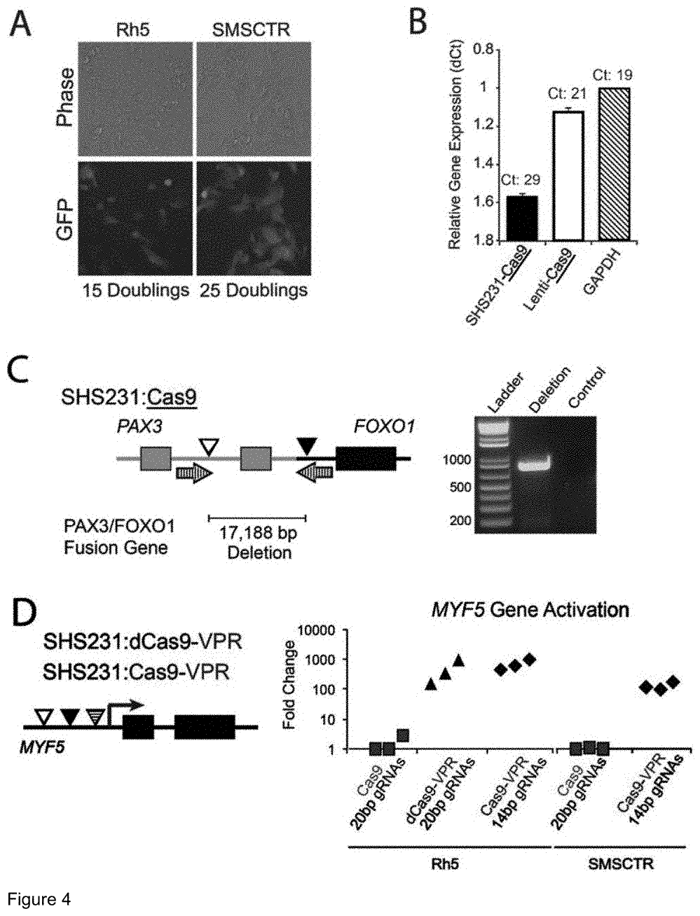

Transgene stability following SHS231 integration was analyzed by selection and GFP expression (FIG. 4A). Time-course imaging of GFP fluorescence was performed using an EVOS imaging system (Life Technologies), and the continued expression of SHS231 transgene-encoded Cas9 was quantified by qRT-PCR SYBR green fluorescence on an CFX96 quantitative PCR (qPCR) machine (Cas9 gFwd; 5′-CCCAAGAGGAACAGCGATAAG-3′ (SEQ ID NO: 38), Cas9 qRev; 5′-CCACCACCAGCACAGAATAG-3′ (SEQ ID NO: 39): BioRad, Hercules, Calif.). The functional activity of SHS-integrated, transgene-encoded Cas9 protein to promote additional rounds of gene editing was demonstrated by lentiviral transduction and expression of dual gRNAs specific for the PAX3/FOXO1 fusion oncogene contained in rhabdomyosarcoma cell line Rh30 (FIG. 4B; P/F gRNA1: 5′-GATCAATAGATGCTCCTGA-3′ (SEQ ID NO: 40), P/F gRNA2: 5′-GACCTTGTTTTATGTGTACA-3′ (SEQ ID NO: 41)). The resulting 17.2 kb gDNA-directed deletions were detected using PCR amplification of the region spanning the target gDNA deletion site (FIG. 4B; P/F Fwd: 5′-AGGTTGTCCTGAACGTACCTATCAC-3′ (SEQ ID NO: 42) and P/F Rev: 5′-TGCTTCTCCGACACCCCTAATCT-3′ (SEQ ID NO: 43); 885 bp).

The functional competence of SHS231 transgene-encoded proteins was further demonstrated using two expression cassettes for the Cas9-based transcription activator proteins dCas9-VPR or Cas9-VPR. Lentiviral expression of dual or triple Cas9 gRNAs was used to target these transactivators to the endogenous, silent MYFS gene in Rh5 and SMSCTR cells. The MYF5 promoter activating gRNAs for dCas9-VPR were gRNA1A, 5′-GATTCCTCACGCCCAGGAT-3′ (SEQ ID NO: 44); gRNA2A, 5′-GTTTGTCCAGACAGCCCCCG-3′ (SEQ ID NO: 45); and gRNA3A, 5′-GTTTCACACAAAAGTGACCA-3′ (SEQ ID NO: 46). The corresponding truncated activating Cas9-VPR gRNAs targeting the MYFS promoter region were tgRNA1A: 5′-GATAGGCTAAAACAA-3′ (SEQ ID NO: 47) and tgRNA2A: 5′-GTGCCTGGCCACTG-3′ (SEQ ID NO: 48). Changes in MYFS gene expression were quantified by SYBR green qRT-PCR using the MYF5-specific primers MYF5 gFwd, 5′-CTGCCCAAGGTGGAGATCCTCA-3′ (SEQ ID NO: 49) and MYFS qRev, 5′-CAGACAGGACTGTTACATTCGGGC-3′ (SEQ ID NO: 50).

The efficiency of SHS231 editing by different endonucleases was determined by co-transfecting two independent RMS cells lines (SMSCTR and RD) with a puromycin-expressing SH231 repair template along with an expression vector for mCrel, for Cas9 nickase (with a single gRNA), or for Cas9 cleavase (with single and dual gRNAs). The RMS cells were also co-transfected with the SHS231 repair template and piggybac transposase plasmid (PB210PA-1, Palo Alto, Calif.), to compare the SHS231 knockin efficiencies of rnCrel and transposase-mediated transgene integration. Two days following transfection, cells were plated into 24 well plates at 3×10e4 cells/well, followed by growth in the presence of puromycin (2.5 μg/ml) for 10 days. Cells were then fixed with 2% paraformaldahyde, stained with 0.5% crystal violet and imaged on a Nikon SMZ-745 stereomicroscope to quantify cell number by counting crystal violet stained pixels using imageJ software (NIH).

### RESULTS

New Human Safe Harbor Site Identification

Our BLAST search of 128 predicted highly cleavable mCrel target site variants revealed 27 unique mCrel target sites matches in the human genome (FIGS. 1A and 1B). A majority of these target sites were found only once (24/27, 89%), while the remaining 3 were represented 2, 3 or 6 times in the human genome for a total of 35 target site matches at different genomic locations (FIG. 1C, Table 2). One of these target sites was a perfect match to a mCrel target site variant (a 20/20 bp match, or 100% identity), whereas the other hits differed by 1 bp (i.e., were 19/20 bp matches or 95% identical) to a query site sequence. The 35 mCrel target sites were located on 16 of the 23 human chromosome pairs including the X chromosome, and covered nearly half of all chromosome arms (23 of 48; FIG. 1C, Table 2).

All 35 new target sites, together with the three canonical human SHS AAVS1, CCR5 and hROSA26, were next evaluated using 8 safety, functional and accessibility criteria in addition to site uniqueness (Table 1 and 2). Among our 35 newly identified sites, 25 (or 71%) fulfilled more than half (≥5/9) of our SHS criteria, as did the AAVS1 and CCR5 canonical human SHS (Table 2). When we examined safety criteria alone (SHS criteria 1-6 in Table 1), 21/35 (60%) of our target sites met ≥4 of 6 criteria, with three (SHS231, 233 and 303) matching all 6 safety criteria.

In contrast, the widely used human SHS AAVS1, CCR5 and hROSA26 each matched only 3 of 6 safety criteria (Table 2). This site assessment was more extensive than previous attempts and made systematic use of genomic data that together, allowed us to rank-order both newly identified and canonical SHS for potential utility and experimental verifications (Table 2).

Genetic variation between individuals has the potential to complicate or disrupt the editing of SHS as well as other genomic regions, In order to assess the potential magnitude of this problem, we assessed all 35 of our new SHS for copy number and basepair-level genetic variation. None of our target sites was located in a copy number-variable region of the human genome, though we did identify base pair-level genetic variation in 10 of our 35 mCrel target sites in whole genome sequencing data generated as part of the 1000 Genomes Project (21). This site-specific base-pair variation was restricted to single nucleotide polymorphic variants (SNPs or SNVs); no indels were identified, Four SHS contained potential mCrel cleavage-inactivating SNP variants: SHS255 on chromosome 5 (variant frequency=0.5041), SHS301 on chromosome 7 (variant frequency=0.2234), SHS293 on chromosome 8 (variant frequency=0.0037) and SHS297 on chromosome 17 (variant frequency=0.0751). All four SNPs were predicted to strongly suppress mCrel cleavage efficiency by ≥70% (FIG. 1B, Table 4). Of note, among individuals analyzed as part of the 1KGP, 80% lacked any SNP variants in any of our 35 target sites including SHS231, and 94% had all 35 target sites predicted fully mCrel-cleavage sensitive despite the presence of one or more permissive base-pair variant SNP (Table 4).

TABLE 4
Nucleotide sequence variants in mCrel genomic target sites,
together with predicted effect on mCrel cleavage sensitivity

| Site | | | | SNV | | | Cre | |
|---|---|---|---|---|---|---|---|---|
| ID | Chr | Start | End | Position | SNP | Frequency | position | Effect |
| 323 | 1 | 152360840 | 152360859 | 152360844 | C/T | 0.000457875 | G @ +6 | 0.81 |
| | | | | | | | (rev) | |
| 229 | 2 | 45708354 | 45708373 | 45708365 | C/T | 0.002289377 | C @ +2 | 0.99 |
| 283 | 4 | 37769238 | 37769257 | 37769243 | A/G | 0.000457875 | A @ −5 | 0.69 |
| | | | | 37769246 | A/G | 0.000457875 | A @ −2 | 1.21 |
| 315 | 5 | 7577728 | 7577747 | 7577738 | A/G | 0.007326007 | C @ −1 | 0.59 |
| | | | | | | | (rev) | |
| 255 | 5 | 19069307 | 19069326 | 19069307 | A/G | 0.504120879 | G @ −10 | 0.28 |
| 305 | 5 | 159922029 | 159922048 | 159922040 | C/T | 0.009157509 | G @ −2 | 1.00 |
| | | | | | | | (rev) | |
| 301 | 7 | 113327685 | 113327704 | 113327699 | C/T | 0.223443223 | T @ 5 | 0.21 |
| 257 | 7 | 138809594 | 138809613 | 138809604 | A/G | 0.000457875 | C @ −1 | 0.59 |
| | | | | | | | (rev) | |
| 293 | 8 | 40727927 | 40727946 | 40727939 | A/G | 0.003663004 | T @ −3 | 0.17 |
| | | | | | | | (rev) | |
| 297 | 17 | 14810285 | 14810304 | 14810291 | C/T | 0.075091575 | C @ −4 | 0.16 |

Among 35 newly identified transgene insertion sites 11 had basepair variants within the mCrel target site at the indicated base pair (SNV position column). The location of the SNP variant within the target site sequence by mCrel target site coordinates is shown in column ‘Cre position’ and the predicted effect from the experimentally determined mCrel position-specific weight matrix in FIG. 1A is shown in the ‘Effect’ column. “Effect” indicates the impact of base substitutions on site cleavage sensitivity by mCrel. Scores of 0.9 or greater indicate full sensitivity; 0.3-0.9 partial cleavage sensitivity; and 0.3 or below, cleavage resistance.

Experimental Validation of Potential New Human SHS

In order to experimentally validate the most promising of our potential new SHS, we amplified 28 of the target site regions from the human genome and subjected these to either in vitro mCrel cleavage assays or DNA sequencing. As part of these analyses we identified one polymorphic 108 bp insertion adjacent to SHS231 that was present in a subset of human cell lines. This insertion contained a 35-base poly-T sequence and adjacent short sequence blocks reminiscent of transposable element short tandem duplications, and was found to be an exact match for a segment of an AluYa5 subfamily, SINE-derived repeat of 311 bp that is present in ˜4000 non-redundant copies in the human genome (see: dfam.org/entry/DF0000053). Though located near SHS231, we demonstrate below that this insertion did not affect SHS231 access or editability. A majority of SHS were fully cleavage-sensitive in vitro when compared with the canonical mCrel target site, including single copy SHSs 227, 229, 231, 233, 251, and multi-copy SHSs 253, 255, 257, 259, 263. As noted above, all of the individuals analyzed as part of the 1KGP either lacked any SHS SNP variants (80%), and 94% had all 35 sites predicted fully mCrel-cleavage sensitive (Table 4).

Efficient In Vivo Cleavage and Editing of New SHS by Multiple Genome Editing Nucleases

We assessed the functional competence of potential new SHS by determining their in vivo cleavage sensitivity and ability to be edited by different genome editing nuclease/repair template combinations. These experiments focused on the single copy, highly-ranked chromosome 4q SHS231, and two sites on chromosome 2 that were single copy (SHS229), or as a single copy on chromosome 2 with additional copies on chromosome arms 5p, 7q, 14q, 17q and Xp (SHS253; FIG. 1, Table 2). The in vivo cleavage sensitivity of these and three additional SHS was analyzed by co-expressing mCrel with the TREX2 3′ to 5′ repair exonuclease in human 293T cells, followed by PCR amplification and mCrel digestion of target sites. This experiment was designed to identify a cleavage-resistant target site fraction in nuclease-expressing cells, from which a minimum estimate of in vivo cleavage efficiency can be derived (22).

Five of the 6 SHS assayed in this way, the unique sites SHS227, 229 and 231 and copies of the same target site sequence located on different chromosomes (SHS253, 257 and 263), had increased fractions of mCrel-resistant target site PCR products that ranged from 3.8% to 31.3% when compared with the corresponding SHS-specific PCR product from mock-transfected control cells. The presence of multiple SHS-specific, mCrel-resistant PCR products also provides evidence for the ability of mCrel to cleave-and thus potentially simultaneously edit-multiple target sites in human cells.

In order to determine whether SHS cleavage in viva could catalyze high fidelity homology-dependent repair, we ca-transfected human 293T cells with an expression vector for mCrel, for a CRISPR/Cas9 cleavage/nickase or for a TAL effector nuclease (TALEN) pair together with a SHS-specific repair template containing a loxP site flanked by two different diagnostic restriction sites (FIG. 2). SHS229, 231 and 253 were analyzed following mCrel expression, SHS229 and 231 after CRISPR/Cas9 cleavage/nickase expression, and SHS231 after TALEN expression. FOR amplicons from transfected cells were then subjected to Pvul and SaclI restriction digestion to confirm targeted capture and site-specific integration of the loxP repair template, followed by cloning and DNA sequencing to confirm the structure and fidelity of cleavage-dependent, targeted SHS integration (FIG. 2). The frequency of targeted SHS231 integration events in 293T cells was 4.8% for mCrel/TREX2 (3/63 clones); 6.1% (2/33) for CRISPR/Cas9 nuclease and 16.1% (5/31) for CRISPR/Cas9 nickase; and 1.23% (1/81) for a SHS231-specific TALEN pair (FIG. 2). Infrequent single base substitutions observed in cloned and sequenced loxP inserts were most likely PCR errors introduced by Taq DNA polymerase during site amplifications for cloning and DNA sequencing. Parallel targeted integration assays at SHS229 and 253 showed comparable results (FIG. 6).

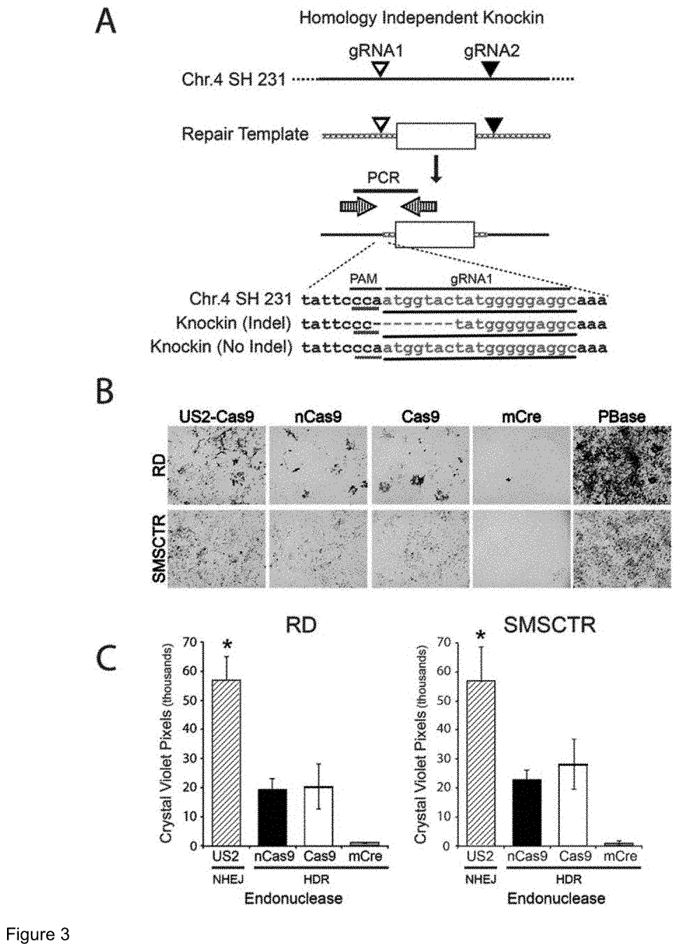

In order to increase SHS engineering efficiency and potentially facilitate the editing in post mitotic cells, we also evaluated SHS231 editing by a potentially homology-independent knockin approach. This strategy used Cas9-mediated cleavage of the repair template and genomic SHS target locus (i.e., using dual gRNAs; US2-Cas9) to promote potential repair with transgene integration by NHEJ-mediated repair mechanisms (32,33) (FIG. 3A). While indel mutations can be introduced during NHEJ-mediated repair in the cleaved target locus and repair template, this is not a serious concern since our SHS were specifically identified to contain no functional genomic elements and the repair template cleavage site did not inactivate the encoded transgene(s). Molecular analysis of SHS231 integration events by amplification, cloning and sequencing of the 5′ SHS231 integration site identified both direct fusion events (no indels), as well as the expected short indel mutations at the gRNA cleavage site (FIG. 3A), evidence compatible with NHEJ-mediated integration. The efficiency of dual gRNA Cas9 cleavage-mediated editing of the SHS231 locus was compared to the Cas9 nickase, cleavage and rnCrel-mediated HDR approaches by co-transfection of each endonuclease with a repair template expressing puromycin (FIG. 3B-C, FIG. 5). The efficiencies of these endonucleases was also compared to random integration of the repair template using a piggybac transposon, since the repair template contained piggybac terminal repeat sequences flanking the transgene cassette. This experiment was performed in two independent RMS cells lines (RD and SMSCTR), where the putative homology-independent insertion or knockin of the puromycin repair template was 2-fold higher when compared to HDR-mediated insertion. Neither of these approaches, however, was as efficient as random integration by piggybac-mediated transposition (FIGS. 3B and 3C).

Characterization of stability, expression, and functionality of SHS231 integrated genes

The functional utility of any SHS depends critically upon persistent marking and/or SHS-specific gene expression after site editing. In order to assess this key SHS functional requirement, we analyzed the expression of several different transgene cassettes that had been integrated into the chromosome 4 SHS231. SHS transgene expression stability was assessed by integrating, and then following the expression of, a SHS231 GFP reporter cassette in two independent RMS cells lines (SMSCTR and Rh5) where transgene insertion was mediated by putative homology-independent editing. When GFP transgene expression was followed over several weeks (i.e., over 45 days) in the absence of antibiotic selection, we observed no significant decrease in GFP expression after 15 population doublings (Rh5) or 25 population doublings (SMSCTR; FIG. 4A). These results highlight the stable nature of transgene integration and expression from SHS231, over usefully long periods of time in mitotically dividing cells.

We next determined whether SHS231-integrated, Cas9-derived transgenes were not only persistently expressed but retained theft intended functions. Stable Cas9-expressing cell lines are a convenient starting point for a growing range of Cas9-enabled methods to study gene structure, function or to enable genetic screens. We observed readily detectable Cas9 expression from SHS231 knockin transgenes that was comparable to cells super-infected with high titer lentivirus to express Cas9 protein, or to the expression of endogenous GAPDH protein (FIG. 4B). The functional competence of SHS231-expressed Cas9 protein was further demonstrated in Rh30 RMS cells by transducing cells with a lentivirus expressing two gRNAs targeting a PAX3/FOXO1 fusion oncogene contained in Rh30 (FIG. 4C). Efficient generation of the predicted 17,188 bp gDNA-targeted deletion in PAX3/FOXO1 was readily detected by PCR amplification of gRNA-transduced cell pools using primers that flanked the PAX3/FOXO1 gRNA target sites (FIG. 4C).

In a third series of SHS functional validation experiments, we integrated transgene cassettes in SHS231 that expressed chimeric Cas9-derived transcriptional activators dCas9-VPR or Cas9-VPR by Cas9-mediated knockin. VPR is a tripartite transcription factor consisting of VP64, P65 and Rta transactivation domains (34). Fusion of this transcription factor to the C-terminus of the Cas9 protein generates a potent, programmable transcriptional activator (dCas9-VPR or Cas9-VPR) (34). Each SHS231 RMS cell line expressing dCas9-VPR or Cas9-VPR was then transduced with a lentivirus expressing 2 or 3 gRNAs targeting the promoter region of the MYF5 gene (FIG. 4D). MYFS is typically not expressed or expressed at very low levels in many RMS cells, and therefore is a good candidate for measuring gRNA-targeted Cas9-VPR-mediated gene activation. We found that both full length (20bp) and truncated (14 bp) gRNAs promoted robust Cas9-VPR-dependent MYFS gene activation in both of the RMS cell lines tested (FIG. 4D).

These results collectively demonstrate efficient editing of a newly defined human safe harbor site, and the stable expression of functionally useful SHS231-integrated transgenes encoding GFP and Cas9 protein variants. Moreover, we demonstrate the ability of these proteins to drive additional useful outcomes including genome editing with the promotion of large deletions in a PAX3/FOXO1 fusion oncogene, and induced expression of the MYFS gene that is normally silent in RMS cells. The SHS231-specific targeting vectors used in these experiments have been assembled into a SHS231-specific ‘toolkit’ to enable facile editing of the highly-ranked SHS231 in a wide range of human cell types (FIG. 5, Table 5). This SHS231 toolkit is available from Addgene (Addgene, Cambridge, Mass.), and includes both Cas9 and dCas9-based expression cassettes, as well as GFP and RFP reporter constructs with puromycin, hygromycin and blasticidin selectable markers. All of the expression vector transgenes included in this set are driven by the human EF-1α promoter and contain additional attP sites to serve as ‘landing pads’ for ϕC31 and Bxb1-mediated, high efficiency SHS transgene insertion.

TABLE 5
Human chromosome 4 SHS231 genome editing toolkit

| | Description | Addgene | Description |
|---|---|---|---|
| 1 | pSH231-EF1- | 115143 | PuroR expressing |
| | Puro | | SH231 vector |
| 2 | pSH231-EF1- | 115144 | GFP-T2A-HygroR |
| | GFP-HYGRO | | expressing SH231 |
| | | | vector |
| 3 | pSH231-EF1- | 115145 | RFP-T2A-HygroR |
| | RFP-HYGRO | | expressing SH231 |
| | | | vector |
| 4 | pSH231-EFS- | 115146 | Cas9-T2A-BlastR |
| | Cas9-BlastR | | SH231 vector |
| 5 | pSH231-EF1- | 115147 | BlastR-T2A-Cas9- |
| | BLST-Cas9-VPR | | VPR SH231 vector |
| 6 | pSH231-EF1- | 115148 | BlastR-T2A-dCas9- |
| | BLST-dCas9-VPR | | VPR SH231 vector |
| 7 | pSH231-Bx- | 115149 | Base pSH231 vector |
| | GFP-C31 | | containing SH231 |
| | | | homology arms and |
| | | | Bxb1 and FC31 attP |
| | | | landing pads flanking |
| | | | a multiple cloning |
| | | | site. |
| 8 | pUS2- | 115150 | Cas9-GFP expression |
| | SH231 | | vector for targeted |
| | | | integration of repair |
| | | | templates into the |
| | | | safe harbor 231 site. |

Discussion

Only a small number of SHS are in wide use in human cells. These were originally identified by serendipity (AAVS1, CCR5) or by their similarity to SHS in other organisms (e.g., hROSA26). In order to address the continuing need for additional well-validated human SHS to enable a broader range of basic and translational science applications, we used a systematic approach to identify and evaluate 35 potential new SHS in the human genome. These new SHS cover a substantial fraction of the human genome: 16 of 23 chromosomes including the X chromosome, with SHS on 23 of 48 chromosome arms (FIG. 1). These potential new SHS were assessed and rank-ordered as potential ‘safe harbors’ using both previously suggested criteria (e.g., 17) and additional more recently available human genome-scale structural, genetic and regulatory data (e.g., ENCODE data (18)). Over half of our new SHS (20135, or 57%) met 4 of our 6 core safety criteria (Tables 1 and 2), in contrast to the widely used human AAVS1, CCR5 and hROSA26 SHS that each met 3 or fewer of these core safety criteria (Table 2).

All 35 of these newly identified SHS contained a site-anchoring 20 bp mCrel nuclease cleavage site, and thus can be immediately targeted either singly or in multiplexed fashion using this small, easily vectorized homing endonuclease together with SHS-specific repair templates (7-9). All of these SHS can also be targeted by virtue of overlapping or adjacent Cas9 and TALEN target sites, as we demonstrated for three different sites located on chromosomes 2 and 4. Of note, human population genomic data indicate that few of these 35 new human SHS harbor any genetic variation that would prevent their use for mCrel, Cas9 or TALEN-mediated editing in human cells or cell lines.

As part of the experimental validation of a subset of these new human SHS, we demonstrated both Cas9 nickase and cleavage-dependent editing, and efficient editing of the chromosome 4 SHS231 by both homology-dependent and likely homology-independent, NHEJ-mediated mechanisms. High efficiency, homology-independent transgene integration strategies in which both template and target locus are cleaved may facilitate higher efficiency site-specific editing while taking advantage of the less stringent requirements for editing than endogenous open reading frame editing by higher fidelity homology-dependent approaches. Thus a dual-cleavage knockin approach may facilitate the efficient generation of cell populations with virtually identical, site-specific transgene insertions. This approach could in many instances eliminate the time and expense of isolating multiple cell clones, while retaining the natural heterogeneity found in the human cells and cell lines most often used to study and model biological systems. Dual-cleavage knockin strategies also have the potential to open many non-dividing cell types to efficient genome engineering, in contrast to homology-dependent pathways that can only be efficiently used in dividing cells.

Several aspects of our newly defined SHS remain to be explored and/or optimized. While we have thus far extensively validated only a subset of our sites (SHS231, 229 and 253; FIG. 1), we anticipate these sites will be representative of most or all of our other newly identified SHS in different cell types, Most notable among these results was targeted transgene insertion with persistent expression from SHS231 of useful transgene-encoded proteins such as Cas9 variants, selectable markers and fluorescent proteins. Stable transgene expression is a key requirement for SHS, and thus will need to be further verified to identify SHS-specific variables that might affect SHS editing and transgene expression in different cell types (see, e.g., Daboussi et al., 2012 (38)). Should site-specific problems arise, the substantial expansion of useful new human SHS identified here may provide ready experimental alternatives.

The efficiency of SHS-targeted editing can likely also be further optimized. Important variables include cell type-specific gene transfer efficiencies; repair template type (single-vs double-stranded), and the length and degree of nucleotide sequence identity between the repair template and target site flanking sequences, The highest efficiency of homology-directed repair can in most instances be promoted by incorporating >200bp of perfect DNA sequence identity between a SHS and donor repair template arms (39-42). Thus target site characterization in cell types of interest is an important part of any homology-dependent editing optimization workflow, in order to identify potentially confounding issues such as the variable SIN E/Alu-derived short insertion we identified near the SHS231 site in a subset of cell lines. This type of unanticipated finding, once identified, can be readily incorporated into the construction of repair templates where long, flanking homology arms are desirable or required.

The new SHS identified here expand by an order of magnitude the number of human SHS that can be used for human genome editing and engineering applications. The SHS assessment and scoring strategy we used was more comprehensive that previous efforts, and can be further modified to incorporate new or application-specific SHS scoring criteria. For example, the growing number of apparently dispensable human genes (6,43) offers one rich source of potential new human SHS. These human gene ‘knockout’ lists can be supplemented with complementary lists of essential or high fitness human genes, to focus on genomic regions to target or avoid as part of genome engineering projects (44-46). The characterization of additional new human SHS and the development of SHS-specific reagents such as our SHS231 ‘toolbox’ should provide practically useful tools to enable a wide range of basic as well as translational human genome engineering applications.

### Example 2

### Human Genomic Safe Harbor Site Region with Inclusion/Exclusion Criteria and Zones

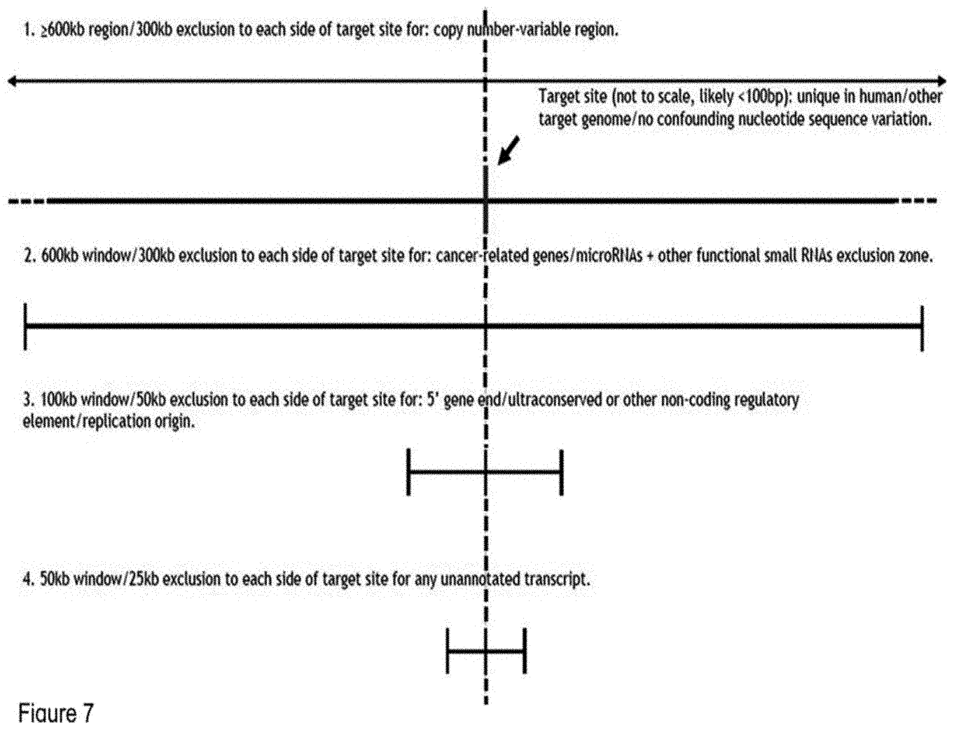

An exemplary diagram illustrating implementation of a selection process as described herein is provided in FIG. 7. Criteria for selection can first be identified and prioritized as suggested in Table 1, based on the intended use. The regions surrounding putative target sites can then be examined in the UCSC Genome Browser (genome.ucsc.edu/cgi-bin/hgTracks?hgt_tSearch=track+search) using the corresponding track source indicated in Table 1.

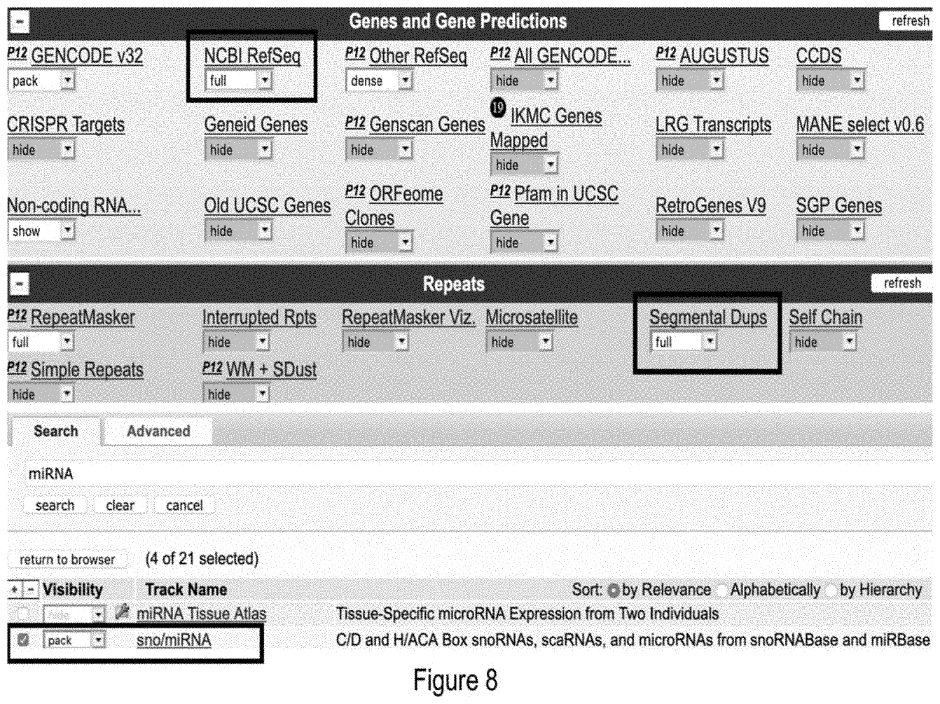

In this example, one first examines 300 kb to each side of a putative target site (typically less then 100 bp and unique in target genome, with no confounding nucleotide sequence variation), for exclusion of copy number-variable region, and then for exclusion of cancer-related genes, microRNAs, and other functional small RNAs. FIG. 8 is a screenshot image of the display in UCSC Genome Browser from which one can activate the corresponding tracks. Genes within the 600 kb region (300 kb on either side of putative target site) can be cross-referenced against the current Cancer Gene Census (CGC) list available at cancersangerac.uk/census. A search of “Sno/miRNA” can identify all microRNAs (miRNA). Likewise, “RefSeq Curated” can be used to identify all genes and 5′ ends of annotated genes, and “Segmental Dups” can be used to identify copy number variable regions.

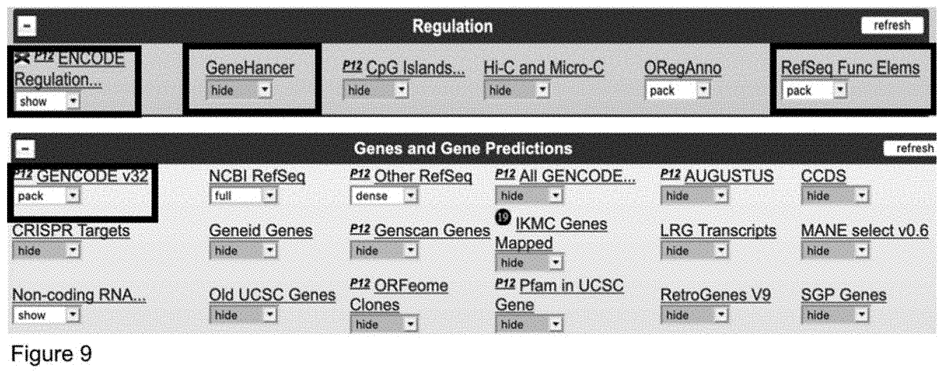

As illustrated in the FIG. 9 screenshot image of the additional displays in the UCSC Genome Browser, further tracks can be activated, such as “GeneHancer” to identify ultra-conserved regions, “RefSeq Func Elems” to identify replication origins and non-coding regulatory elements, “GENCODEv32” to identify all transcripts (annotated and un-annotated), and “ENCODE regulation” to identify regions of open chromatin.

Use of these criteria is then scored via the 3 score system described above. For example, 2 indicates perfect match/in agreement; 1 is a partial match; and 0 signifies a fail for a specific criterion identified in the targeted window when the specified track is active in the browser.

### REFERENCES

1. DeKelver R C, Choi V M, Moehle E A, et al. Functional genomics, proteomics, and regulatory DNA analysis in isogenic settings using zinc finger nuclease-driven transgenesis into a safe harbor locus in the human genome. Genome Res 2010;20:1133-1142.

2. Mali P, Yang L, Esvelt K M, et al. RNA-guided human genome engineering via Cas9. Science 2013;339:823-826.

3. Inion S, Luche H, Gadue P, et al. Identification and targeting of the ROSA26 locus in human embryonic stem cells. Nat Biotechnol 2007;25;1477-1482.

4. Li L, Krymskaya L, Wang J, et al. Genomic editing of the HIV-1 coreceptor CCRS in adult hematopoietic stem and progenitor cells using zinc finger nucleases. Mol Ther 2013;21:1259-1269.

5. Lombardo A, Genovese P, Beausejour C M, et al. Gene editing in human stern cells using zinc finger nucleases and integrase-defective lentiviral vector delivery. Nat Biotechnol 2007;25:1298-1306.

6. MacArthur D G, Balasubramanian S, Frankish A, et al. A systematic survey of loss-of-function variants in human protein-coding genes. Science 2012;335:823-828.

7. Jurica M S, Monnat R J, Stoddard B L. DNA recognition and cleavage by the LAGLIDADG horning endonuclease I-Cre I. Mol Cell 1998;2:469-476.

8. Li H, Pellenz S, Ulge U, et al. Generation of single-chain LAGLIDADG homing endonucleases from native homodimeric precursor proteins. Nucleic Acids Res 2009;37:1650-1662.

9. Heath P J, Stephens K M, Monnat R J, et al. The structure of I-Crel, a group I intron-encoded homing endonuclease. Nat Struct Biol 1997;4:468-476.

10. Hinson A R P, Jones R, Crose L E S, et al. Human rhabdomyosarcoma cell lines for rhabdomyosarcoma research: Utility and pitfalls. Front Oncol;3. Epub ahead of print Jul. 17, 2013. doi: 10,3389/fonc.2013.00183.

11. Argast G M, Stephens K M, Emond M J, et al. I-Ppol and I-Crel homing site sequence degeneracy determined by random mutagenesis and sequential in vitro enrichment. J Mol Biol 1998;280:345-353.

12. Friedman J I, Li H, Monnat R J. Quantifying the information content of homing endonuclease target sites by single base pair profiling. In: Homing Endonucleases. Humana Press, Totowa, N.J.; pp. 135-149.

13. Li H, Monnat R J. Horning endonuclease target site specificity defined by sequential enrichment and next-generation sequencing of highly complex target site libraries. In: Homing Endonucleases. Humana Press, Totowa, N.J.; pp. 151-163.

14. Li H, Ulge U Y, Hovde B T, et al. Comprehensive horning endonuclease target site specificity profiling reveals evolutionary constraints and enables genome engineering applications. Nucleic Acids Res 2012;40:2587-2598.

15. Pellenz S, Monnat R J. Identification and analysis of genomic homing endonuclease target sites, In: Horning Endonucleases. Humana Press, Totowa, N.J.; pp. 245-264.

16. Ulge U Y, Baker D A, Monnat R J. Comprehensive computational design of mCrel homing endonuclease cleavage specificity for genome engineering. Nucleic Acids Res 2011;39:4330-4339.

17. Sadelain M, Papapetrou E P, Bushman F D. Safe harbours for the integration of new DNA in the human genome. Nat Rev Cancer 2012;12:51-58.

18. Consortium TEP. An integrated encyclopedia of DNA elements in the human genome. Nature 2012;489:57-74.

19. Kuhn R M, Haussler D, Kent W J. The UCSC genome browser and associated tools. Brief Bioinform 2013;14:144-161.

20. Meyer L R, Zweig A S, Hinrichs A S, et al. The UCSC genome browser database: extensions and updates 2013. Nucleic Acids Res 2013;41:D64-D69.

21. Consortium T 1000 GP. An integrated map of genetic variation from 1,092 human genomes. Nature 2012;491:56-65.

22. Certo M T, Gwiazda K S, Kuhar R, et al. Coupling endonucleases with DNA end-processing enzymes to drive gene disruption. Nat Methods 2012;9:973-975.

23. Chen C, Okayama H. High-efficiency transformation of mammalian cells by plasmid DNA. Mol Cell Biol 1987;7:2745-2752.

24. Dull T, Zufferey R, Kelly M, et al. A third-generation lentivirus vector with a conditional packaging system. J Virol 1998;72:8463-8471.

25. Szymczak-Workman A L, Vignali K M, Vignali D A A. Design and construction of 2A peptide-linked multicistronic vectors. Cold Spring Harb Protoc 2012;2012:199-204.

26. Cermak T, Doyle E L, Christian M, et al. Efficient design and assembly of custom TALEN and other TAL effector-based constructs for DNA targeting. Nucleic Acids Res 2011;39;e82-e82.

27. Doyle E L, Booher N J, Standage D S, et al. TAL Effector-Nucleotide Targeter (TALE-NT) 2.0: tools for TAL effector design and target prediction. Nucleic Acids Res 2012;40:W117-W122.

28. Boissel S, Jarjour J, Astrakhan A, et al, megaTALs: a rare-cleaving nuclease architecture for therapeutic genome engineering. Nucleic Acids Res 2014;42:2591-2601.

29. Cong L, Ran F A, Cox D, et al. Multiplex genome engineering using CRISPR!Cas systems. Science 2013;339:819-823.

30. Hsu P D, Scott D A, Weinstein J A, et al. DNA targeting specificity of RNA-guided Cas9 nucleases. Nat Biotechnol 2013;31:827-832.

31. Phelps M P, Bailey J N, Vleeshouwer-Neumann T, et al. CRISPR screen identifies the NCOR/HDAC3 complex as a major suppressor of differentiation in rhabdomyosarcoma. Proc Natl Acad Sci 2016;201610270.

32. Auer T O, Duroure K, Concordet J-P, et al. CRISPR/Cas9-mediated conversion of eGFP-into Gal4-transgenic lines in zebrafish. Nat Protoc 2014;9:2823-2840.

33. Suzuki K, Tsunekawa Y, Hernandez-Benitez R, et al. In vivo genome editing via CRISPR/Cas9 mediated homology-independent targeted integration. Nature 2016;540:144-149.

34. Chavez A, Scheiman J, Vora S, et al. Highly efficient Cas9-mediated transcriptional programming. Nat Methods 2015;12:326-328.

35. He C, Gouble A, Bourdel A, et al. Lentiviral protein delivery of meganucleases in human cells mediates gene targeting and alleviates toxicity. Gene Ther 2014;21:759-766,

36. Monnat R J, Hackmann A F M, Cantrell M A. Generation of highly site-specific DNA double-strand breaks in human cells by the homing endonucleases I-Ppol and I-Crel. Biochem Biophys Res Commun 1999;255:88-93.

37. Smith A M, Takeuchi R, Pellenz S, et al. Generation of a nicking enzyme that stimulates site-specific gene conversion from the I-Anil LAGLIDADG homing endonuclease. Proc Natl Acad Sci 2009;106:5099-5104.

38. Daboussi F, Zaslayskiy M, Poirot L, et al. Chromosomal context and epigenetic mechanisms control the efficacy of genome editing by rare-cutting designer endonucleases. Nucleic Acids Res 2012;40:6367-6379.

39. Donoho G, Jasin M, Berg P. Analysis of gene targeting and intrachromosomal homologous recombination stimulated by genomic double-strand breaks in mouse embryonic stem cells. Mol Cell Biol 1998;18:4070-4078.

40. Jasin M, Rothstein R. Repair of strand breaks by homologous recombination. Cold Spring Harb Perspect Biol 2013;5:a012740.

41. LaRocque JR, Jasin M. Mechanisms of recombination between diverged sequences in wild-type and BLM-deficient mouse and human cells. Mol Cell Biol 2010;30:1887-1897.

42. Renkawitz J, Lademann C A, Jentsch S. Mechanisms and principles of homology search during recombination. Nat Rev Mol Cell Biol 2014;15:369-383.

43. Saleheen D, Natarajan P, Armean I M, et al. Human knockouts and phenotypic analysis in a cohort with a high rate of consanguinity. Nature 2017;544:235-239.

44. Wang T, Wei J J, Sabatini D M, et al. Genetic Screens in Human Cells Using the CRISPR-Cas9 System, Science 2014;343:80-84.

45. Blomen V A, Májek P, Jae L T, et al. Gene essentiality and synthetic lethality in haploid human cells. Science 2015;350:1092-1096.

46. Hart T, Chandrashekhar M, Aregger M, et al. High-Resolution CRISPR Screens Reveal Fitness Genes and Genotype-Specific Cancer Liabilities. Cell 2015;163:1515-1526.

Throughout this application various publications are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method of selecting genomic target sites for a desired genome engineering application, the method comprising:
(a) seeding a search matrix with putative genomic target site nucleotide sequences having defined target specificity and degeneracy appropriate for the desired genome engineering application;
(b) searching a specified version of a genome reference sequence to identify sites that share at least 95% identity with potential target sites defined in step (a); and
(c) selecting sites identified in (b) for which satisfaction of the following predefined criteria can be determined:
(i) unique in the reference genome sequence (no more than 1 site per haploid genome);
(ii) not in copy number-variable region;
(iii) target site does not contain nucleotide sequence or other genomic variation that would impede successful targeting;
(iv) at least 25 kilobases (kb) from an unannotated transcript;
(v) at least 50 kb from a 5′ gene end;
(vi) at least 50 kb from an ultra-conserved genomic region, enhancer, or other noncoding regulatory region;
(vii) at least 50 kb from a replication origin;
(viii) at least 300 kb from any microRNA or other functionally annotated small RNA;
(ix) at least 300 kb from a cancer-related gene.

2. The method of claim 1, further comprising:
(d) ranking the putative genomic target sites selected in step (c) according to the desired genome engineering application;
(e) validating target site presence in a targeted genomic sequence, cleavage efficiency of the site(s), and targeted insertion efficiency and fidelity of the transgene at the identified genomic target sites ranked in step (d); and, optionally,
(f) assessing genomic or functional effects of desired genome engineering at selected sites to identify sites to be deselected due to off-target effects.

3. The method of claim 1, wherein the desired genome engineering application is transgene insertion, functional gene editing, gene or chromosomal location-specific structural modification, cell marking, gene activation, or gene repression.

4. The method of claim 1, wherein the search matrix comprises a position weight matrix (PWM).

5. The method of claim 1, wherein the selecting comprises selecting sites that satisfy each of the predefined criteria of (c).

6. The method of claim 2, wherein the ranking of step (d) assigns preference to criteria associated with safety, functional silence, and accessibility, respectively.

7. The method of claim 2, wherein the ranking of step (d) is based on searching genome browser data.

8. The method of claim 7, wherein the genome browser data are aggregated at and obtained from UCSC Genome Browser and/or Ensembl Genome Browser.

9. The method of claim 2, wherein the ranking of step (d) is based on scoring genomic target sites that satisfy the set of predetermined criteria of step (c).

10. The method of claim 2, wherein the ranking of step (d) is based on assessment of copy number variation and/or base pair level variation in sites identified in (b).

11. The method of claim 10, wherein the assessment comprises a survey of human population genomic variation data.

12. The method of any of claim 2, wherein the validating is performed in silico.

13. The method of claim 2, wherein the validating for site presence and cleavage efficiency of step (d) comprises polymerase chain reaction (PCR) amplification of targeted sites and cleavage testing.

14. The method of claim 2, wherein the validating of step (e) comprises homology-dependent recombination (HDR) and/or non-homologous DNA end joining (NHEJ) and/or non-cleavage dependent base or prime editing.

15. The method of claim 2, wherein the validating of step (e) comprises DNA sequencing, transgene expression and/or functional assays for a minimum of 10 cell population doublings to assess stability of transgene insertion and expression.

16. The method of claim 2, wherein the assessing of step (f) comprises genomic or functional assessments.

17. The method of claim 1, further comprising ranking potential genomic target sites for desired genome engineering comprising assigning a weighted score to each of (i)-(ix) and ranking the potential genomic target sites in order of the assigned weighted score.

18. The method of claim 1, further comprising generating a list of genomic target sites selected by the method.

19. The method of claim 18, wherein the method is implemented on a computer, the computer having one or more processors and a memory storing one or more programs for execution by the one or more processors, the one or more programs including instructions for performing steps (a) to (c).

20. The method of claim 19, wherein the seeding of step (a) comprises receiving by the processor instructions to load a target genome sequence and a list of putative target site sequences, wherein the target genome sequence is specified by a genome browser or other defined genome source files, and wherein the list of putative target site sequences is pre-defined list or generated from an algorithm.

21. The method of claim 19, wherein the searching of step (b) comprises receiving by the processor instructions to exclude target sites containing insertions or deletions with respect to the reference sequence.

22. The method of claim 19, wherein the selecting of step (c) comprises receiving instructions (i) to identify one or more criteria selected from: copy number variable regions, microRNAs, ultra-conserved regions, replication origins, non-coding regulatory elements, annotated transcripts, unannotated transcripts, and regions of open chromatin, and (ii) to assign a score indicative of the identified criteria.

23. A method of producing a targeting construct for insertion of a transgene into a genomic site comprising:
(a) selecting a genomic targeting site according to a method described herein; and
(b) synthesizing a construct comprising the transgene flanked by application-specific 5′ and 3′ regulatory sequences, and target site-specific, transgene-flanking homology dependent sequences having sufficient nucleotide sequence homology or identity with the target site sequence to promote transgene insertion into the target site, or homology-independent repair sequence.

24. A targeting construct produced by the method of claim 23.

25. The targeting construct of claim 24, wherein the genomic targeting site of (a) is located on chromosome 2p (SHS229), chromosome 4q (SHS231), or on the short arm of chromosome 2, 5, or X, or on the long arm of chromosome 7, 14, or 17 (SHS253).

26. The targeting construct of claim 24, wherein the genomic targeting site of (a) has the cleavage specificity of the homodimeric I-Crel homing endonuclease and its monomerized derivative mCrel.

27. The targeting construct of claim 24, wherein the genomic targeting site of (a) is selected from SEQ ID NOs: 1-27.

28. The targeting construct of claim 24, wherein the construct targets human chromosome 4 SHS231 and the construct is selected from the group consisting of: pSH231-EF1-Puro, pSH231-EF1-GFP-HYGRO, pSH231-EF1-RFP-HYGRO, pSH231-EFS-Cas9-BlastR, pSH231-EF1-BLST-Cas9-VPR, pSH231-EF1-BLST-dCas9-VPR, pSH231-Bx-GFP-C31, and pUS2-SH231.

29. A cell modified by insertion of targeting construct of claim 24.

30. The cell of claim 29, wherein the cell is modified by insertion of a Bxb1 landing-pad at genomic target site SHS231.

31. A system for selecting genomic target sites for a desired genome engineering application, the system comprising a user device comprising a hardware processor that is programmed to perform the method of claim 1.

32. The system of claim 31, wherein the user device comprises a display screen, and wherein the processor generates and displays on the screen of the user device a list of the genomic target sites selected by the method.

33. The system of claim 31, wherein the user device is hosted at a central location, and wherein the processor transmits the genomic target sites selected by the method to a remote interface.

34. A non-transitory computer-readable medium containing computer executable instructions that, when executed by a processor, cause the processor to perform the method of claim 1.


SEQUENCE LISTING (183 sequences)

SEQ ID NO: 1 - DNA, 20 bp, Homo sapiens
aaaatgtcaggagacatttt

SEQ ID NO: 2 - DNA, 20 bp, Homo sapiens
aaactgtcatgagacatttg

SEQ ID NO: 3 - DNA, 20 bp, Homo sapiens
aaactgtcatgggacagatt

SEQ ID NO: 4 - DNA, 20 bp, Homo sapiens
aaaatgtcatgcgacatttt

SEQ ID NO: 5 - DNA, 20 bp, Homo sapiens
aaactgacataagacagatt

SEQ ID NO: 6 - DNA, 20 bp, Homo sapiens
aaaatgtggtgagacatttt

SEQ ID NO: 7 - DNA, 20 bp, Homo sapiens
aaactgtcataagacagatc

SEQ ID NO: 8 - DNA, 20 bp, Homo sapiens
aaaatgtcatactacagatt

SEQ ID NO: 9 - DNA, 20 bp, Homo sapiens
aaaccgtcgtgatacatttt

SEQ ID NO: 10 - DNA, 20 bp, Homo sapiens
aaactgtcatatgacagatt

SEQ ID NO: 11 - DNA, 20 bp, Homo sapiens
aaaatgtcatgagacagtct

SEQ ID NO: 12 - DNA, 20 bp, Homo sapiens
aaaatgtcaagagacatttt

SEQ ID NO: 13 - DNA, 20 bp, Homo sapiens
aaactgtcaaaagacagatt

SEQ ID NO: 14 - DNA, 20 bp, Homo sapiens
aaactgtcctaagacagttt

SEQ ID NO: 15 - DNA, 20 bp, Homo sapiens
aaaatttcatgagacatttt

SEQ ID NO: 16 - DNA, 20 bp, Homo sapiens
aaaatgtcatgaggcagttt

SEQ ID NO: 17 - DNA, 20 bp, Homo sapiens
aaactgtcataccacagttt

SEQ ID NO: 18 - DNA, 20 bp, Homo sapiens
aaactgtcatacaacagttt

SEQ ID NO: 19 - DNA, 20 bp, Homo sapiens
aaactgacgtaagacagatt

SEQ ID NO: 20 - DNA, 20 bp, Homo sapiens
aaaatgtcctgagacagatt

SEQ ID NO: 21 - DNA, 20 bp, Homo sapiens
aaaaagtcatgagacatttt

SEQ ID NO: 22 - DNA, 20 bp, Homo sapiens
aaactgtagtaagacagatt

SEQ ID NO: 23 - DNA, 20 bp, Homo sapiens
aaaatgtcatgagatatttt

SEQ ID NO: 24 - DNA, 20 bp, Homo sapiens
aaacagtcataagacagatt

SEQ ID NO: 25 - DNA, 20 bp, Homo sapiens
aaactgacatgagacagatt

SEQ ID NO: 26 - DNA, 20 bp, Homo sapiens
aaaatgtcatgagaaagttt

SEQ ID NO: 27 - DNA, 20 bp, Homo sapiens
aaaatgtcataaggcagttt

SEQ ID NO: 28 - DNA, 98 bp, Homo sapiens
cwagggctaaggattaaactgtcatatcgrtcgataacttcgtatagcatacattayacgaagttatccgcggaygacagatyamcaagaggaaagca

SEQ ID NO: 29 - DNA, 30 bp, Homo sapiens
tattcccaatggtactatgggggaggcaaa

SEQ ID NO: 30 - DNA, 20 bp, Homo sapiens
ttggctagggctaaggatta

SEQ ID NO: 31 - DNA, 20 bp, Homo sapiens
tgtatgctttcctcttgtta

SEQ ID NO: 32 - DNA, 22 bp, Homo sapiens
aaaacatttatatactgcgtgg

SEQ ID NO: 33 - DNA, 30 bp, Artificial Sequence
  guide RNA template
ctaatctggacaaaacatttatatactgcg

SEQ ID NO: 34 - DNA, 19 bp, Artificial Sequence
  guide RNA
gcctcccccatagtaccat

SEQ ID NO: 35 - DNA, 20 bp, Artificial Sequence
  guide RNA
gatgtgctcactgagtctga

SEQ ID NO: 36 - DNA, 20 bp, Artificial Sequence
  primer
gaaccagagccacccagttg

SEQ ID NO: 37 - DNA, 24 bp, Artificial Sequence
  primer
gtttgtaccgtacaccactgagac

SEQ ID NO: 38 - DNA, 21 bp, Artificial Sequence
  primer
cccaagaggaacagcgataag

SEQ ID NO: 39 - DNA, 20 bp, Artificial Sequence
  primer
ccaccaccagcacagaatag

SEQ ID NO: 40 - DNA, 19 bp, Artificial Sequence
  primer
gatcaatagatgctcctga

SEQ ID NO: 41 - DNA, 20 bp, Artificial Sequence
  guide RNA
gaccttgttttatgtgtaca

SEQ ID NO: 42 - DNA, 25 bp, Artificial Sequence
  primer
aggttgtcctgaacgtacctatcac

SEQ ID NO: 43 - DNA, 23 bp, Artificial Sequence
  primer
tgcttctccgacacccctaatct

SEQ ID NO: 44 - DNA, 19 bp, Artificial Sequence
  guide RNA
gattcctcacgcccaggat

SEQ ID NO: 45 - DNA, 20 bp, Artificial Sequence
  guide RNA
gtttgtccagacagcccccg

SEQ ID NO: 46 - DNA, 20 bp, Artificial Sequence
  guide RNA
gtttcacacaaaagtgacca

SEQ ID NO: 47 - DNA, 15 bp, Artificial Sequence
  guide RNA
gataggctaaaacaa

SEQ ID NO: 48 - DNA, 14 bp, Artificial Sequence
  guide RNA
gtgcctggccactg

SEQ ID NO: 49 - DNA, 22 bp, Artificial Sequence
  primer
ctgcccaaggtggagatcctca

SEQ ID NO: 50 - DNA, 24 bp, Artificial Sequence
  primer
cagacaggactgttacattcgggc

SEQ ID NO: 51 - DNA, 20 bp, Homo sapiens
aaaacgtcgtgagacagttt

SEQ ID NO: 52 - DNA, 18 bp, Artificial Sequence
  primer
cgaacgccgggttaaggc

SEQ ID NO: 53 - DNA, 18 bp, Artificial Sequence
  primer
cctgccgaatcaactagc

SEQ ID NO: 54 - DNA, 18 bp, Artificial Sequence
  primer
gacaaacccttgtgtcga

SEQ ID NO: 55 - DNA, 20 bp, Artificial Sequence
  primer
gcgcctggcctaaaacattc

SEQ ID NO: 56 - DNA, 20 bp, Artificial Sequence
  primer
tttagtagagaaggggtttc

SEQ ID NO: 57 - DNA, 20 bp, Artificial Sequence
  primer
cttctgatctacactggtcc

SEQ ID NO: 58 - DNA, 20 bp, Artificial Sequence
  primer
ggactggttatctgtctaac

SEQ ID NO: 59 - DNA, 18 bp, Artificial Sequence
  primer
ctcagaggtctggacaca

SEQ ID NO: 60 - DNA, 21 bp, Artificial Sequence
  primer
gctcagatgatcattagcatt

SEQ ID NO: 61 - DNA, 20 bp, Artificial Sequence
  primer
taagaaactgccaccacatc

SEQ ID NO: 62 - DNA, 20 bp, Artificial Sequence
  primer
ccataactcttcctctctct

SEQ ID NO: 63 - DNA, 22 bp, Artificial Sequence
  primer
gaagatgctatgaacgttgtgg

SEQ ID NO: 64 - DNA, 25 bp, Artificial Sequence
  primer
ggcaaataacattctattgtatggg

SEQ ID NO: 65 - DNA, 20 bp, Artificial Sequence
  primer
ccacaacagtaaaccaagtc

SEQ ID NO: 66 - DNA, 20 bp, Artificial Sequence
  primer
cctgtctgatgtcaaggaga

SEQ ID NO: 67 - DNA, 22 bp, Artificial Sequence
  primer
gaagatgctatgaacgttgtgg

SEQ ID NO: 68 - DNA, 50 bp, Artificial Sequence
  primer
ccgcggataacttcgtataatgtatgctatacgaagttatcgatcggcat

SEQ ID NO: 69 - DNA, 50 bp, Artificial Sequence
  primer
cgatcgataacttcgtatagcatacattatacgaagttatccgcggatgc

SEQ ID NO: 70 - DNA, 25 bp, Artificial Sequence
  primer
ggcaaataacattctattgtatggg

SEQ ID NO: 71 - DNA, 21 bp, Artificial Sequence
  primer
gcattctttagtggttgtgaa

SEQ ID NO: 72 - DNA, 20 bp, Artificial Sequence
  primer
tatctgggaaagggtcatct

SEQ ID NO: 73 - DNA, 20 bp, Artificial Sequence
  primer
ccccttgccttgttccattt

SEQ ID NO: 74 - DNA, 20 bp, Artificial Sequence
  primer
gctgctcagctaagcatagc

SEQ ID NO: 75 - DNA, 24 bp, Artificial Sequence
  primer
gaaggagttcagaacacattatcc

SEQ ID NO: 76 - DNA, 20 bp, Artificial Sequence
  primer
gtcacaaattgcattgcatt

SEQ ID NO: 77 - DNA, 20 bp, Artificial Sequence
  primer
cctgcaacaatattctcact

SEQ ID NO: 78 - DNA, 20 bp, Artificial Sequence
  primer
gctgctcagctaagcatagc

SEQ ID NO: 79 - DNA, 50 bp, Artificial Sequence
  primer
ccgcggataacttcgtataatgtatgctatacgaagttatcgatcgatat

SEQ ID NO: 80 - DNA, 50 bp, Artificial Sequence
  primer
cgatcgataacttcgtatagcatacattatacgaagttatccgcggatat

SEQ ID NO: 81 - DNA, 24 bp, Artificial Sequence
  primer
gaaggagttcagaacacattatcc

SEQ ID NO: 82 - DNA, 20 bp, Artificial Sequence
  primer
ggctgaggcaggagaattga

SEQ ID NO: 83 - DNA, 20 bp, Artificial Sequence
  primer
ttacctgaggtcaggtaatc

SEQ ID NO: 84 - DNA, 20 bp, Artificial Sequence
  primer
gcctgacttgatcgttctac

SEQ ID NO: 85 - DNA, 20 bp, Artificial Sequence
  primer
ggagccctaatccaatatgc

SEQ ID NO: 86 - DNA, 22 bp, Artificial Sequence
  primer
ccttatgaatgttttaaatctc

SEQ ID NO: 87 - DNA, 18 bp, Artificial Sequence
  primer
ccagcctgggtgacagag

SEQ ID NO: 88 - DNA, 23 bp, Artificial Sequence
  primer
ggttaagtaaggccaaattaatg

SEQ ID NO: 89 - DNA, 21 bp, Artificial Sequence
  primer
gctgtttttgagaataccctc

SEQ ID NO: 90 - DNA, 20 bp, Artificial Sequence
  primer
tttgcatggcttcttccctc

SEQ ID NO: 91 - DNA, 20 bp, Artificial Sequence
  primer
ttgggaaagttgcttatagg

SEQ ID NO: 92 - DNA, 21 bp, Artificial Sequence
  primer
gtgtcactgaagtgagagcaa

SEQ ID NO: 93 - DNA, 20 bp, Artificial Sequence
  primer
gctgctagagtaagatgagg

SEQ ID NO: 94 - DNA, 20 bp, Artificial Sequence
  primer
cgttaatttcccccatgtat

SEQ ID NO: 95 - DNA, 25 bp, Artificial Sequence
  primer
ggagacagcaagtagcaattgaatg

SEQ ID NO: 96 - DNA, 20 bp, Artificial Sequence
  primer
gccaagcaaatgctggttcc

SEQ ID NO: 97 - DNA, 23 bp, Artificial Sequence
  primer
gctgtcaaatacagttttacaca

SEQ ID NO: 98 - DNA, 21 bp, Artificial Sequence
  primer
cccattggtaagtaatgcatg

SEQ ID NO: 99 - DNA, 25 bp, Artificial Sequence
  primer
ggagacagcaagtagcaattgaatg

SEQ ID NO: 100 - DNA, 49 bp, Artificial Sequence
  primer
ccgcggataacttcgtataatgtatgctatacgaagttatcgatcgtta

SEQ ID NO: 101 - DNA, 50 bp, Artificial Sequence
  primer
cgatcgataacttcgtatagcatacattatacgaagttatccgcggataa

SEQ ID NO: 102 - DNA, 23 bp, Artificial Sequence
  primer
gctgtcaaatacagttttacaca

SEQ ID NO: 103 - DNA, 23 bp, Artificial Sequence
  primer
gacaccttctattatatttcgat

SEQ ID NO: 104 - DNA, 20 bp, Artificial Sequence
  primer
caccagttgaagtaagacct

SEQ ID NO: 105 - DNA, 20 bp, Artificial Sequence
  primer
cagtggcatgatctggagtg

SEQ ID NO: 106 - DNA, 20 bp, Artificial Sequence
  primer
cttctgtgatgccttgaatc

SEQ ID NO: 107 - DNA, 22 bp, Artificial Sequence
  primer
gagaacaaaatccaagcttact

SEQ ID NO: 108 - DNA, 21 bp, Artificial Sequence
  primer
gcctctattcccttctgtacc

SEQ ID NO: 109 - DNA, 20 bp, Artificial Sequence
  primer
tgttcaccatacacttcctc

SEQ ID NO: 110 - DNA, 20 bp, Artificial Sequence
  primer
cagataagcacaaattcacc

SEQ ID NO: 111 - DNA, 22 bp, Artificial Sequence
  primer
ggtaaactatacatcggttggg

SEQ ID NO: 112 - DNA, 19 bp, Artificial Sequence
  primer
ccaaaacctgggtcaccaa

SEQ ID NO: 113 - DNA, 20 bp, Artificial Sequence
  primer
ggcctaggactaggccattc

SEQ ID NO: 114 - DNA, 20 bp, Artificial Sequence
  primer
ggaagagtttaagactggaa

SEQ ID NO: 115 - DNA, 20 bp, Artificial Sequence
  primer
acccttatcttcctagccac

SEQ ID NO: 116 - DNA, 23 bp, Artificial Sequence
  primer
gcttacagtaagagtcaataacc

SEQ ID NO: 117 - DNA, 20 bp, Artificial Sequence
  primer
gcaatcagagtgatcctttc

SEQ ID NO: 118 - DNA, 18 bp, Artificial Sequence
  primer
ccaccgcgcctagctgag

SEQ ID NO: 119 - DNA, 20 bp, Artificial Sequence
  primer
tttttttagtagagacgggg

SEQ ID NO: 120 - DNA, 20 bp, Artificial Sequence
  primer
tggtagatgtggggtttcac

SEQ ID NO: 121 - DNA, 19 bp, Artificial Sequence
  primer
ggattaagcagtgaatggg

SEQ ID NO: 122 - DNA, 20 bp, Artificial Sequence
  primer
ccaccatgtatatccttccc

SEQ ID NO: 123 - DNA, 21 bp, Artificial Sequence
  primer
ggtgtctatcttatgcactgt

SEQ ID NO: 124 - DNA, 20 bp, Artificial Sequence
  primer
gatgctttttgttatggggg

SEQ ID NO: 125 - DNA, 20 bp, Artificial Sequence
  primer
agacaagcttcattcaccac

SEQ ID NO: 126 - DNA, 19 bp, Artificial Sequence
  primer
gaactccactctctgaact

SEQ ID NO: 127 - DNA, 24 bp, Artificial Sequence
  primer
atgatgttcaggataaagtacact

SEQ ID NO: 128 - DNA, 20 bp, Artificial Sequence
  primer
ggcaccattttctcattagc

SEQ ID NO: 129 - DNA, 20 bp, Artificial Sequence
  primer
tggttttgttgtgggagtcc

SEQ ID NO: 130 - DNA, 20 bp, Artificial Sequence
  primer
taacatatagcaaagagggg

SEQ ID NO: 131 - DNA, 20 bp, Artificial Sequence
  primer
tgccctcaagtttcatatgc

SEQ ID NO: 132 - DNA, 20 bp, Artificial Sequence
  primer
gctttctttcctctgggcac

SEQ ID NO: 133 - DNA, 20 bp, Artificial Sequence
  primer
ccatttattgcttgctttcc

SEQ ID NO: 134 - DNA, 20 bp, Artificial Sequence
  primer
ttcagtagagatggggtttc

SEQ ID NO: 135 - DNA, 20 bp, Artificial Sequence
  primer
tactgtgttatgctgacttc

SEQ ID NO: 136 - DNA, 20 bp, Artificial Sequence
  primer
gctcttcctagtctcttctc

SEQ ID NO: 137 - DNA, 20 bp, Artificial Sequence
  primer
ccaccatgcctatctacccc

SEQ ID NO: 138 - DNA, 20 bp, Artificial Sequence
  primer
tccagacaacttttattccc

SEQ ID NO: 139 - DNA, 20 bp, Artificial Sequence
  primer
ataggacacgtaaggaaaga

SEQ ID NO: 140 - DNA, 20 bp, Artificial Sequence
  primer
ttcaatctgtcccaagcatc

SEQ ID NO: 141 - DNA, 20 bp, Artificial Sequence
  primer
agtgtgttcttcagtatcag

SEQ ID NO: 142 - DNA, 20 bp, Artificial Sequence
  primer
tgagagatgtatgtgaggac

SEQ ID NO: 143 - DNA, 20 bp, Artificial Sequence
  primer
ttcttccatgtcactatctg

SEQ ID NO: 144 - DNA, 20 bp, Artificial Sequence
  primer
taatagctacacatgccaac

SEQ ID NO: 145 - DNA, 20 bp, Artificial Sequence
  primer
aaagaggagacaaggttagg

SEQ ID NO: 146 - DNA, 20 bp, Artificial Sequence
  primer
aaggaacagaccatgagaag

SEQ ID NO: 147 - DNA, 20 bp, Artificial Sequence
  primer
ggctgcatcactacattatt

SEQ ID NO: 148 - DNA, 20 bp, Artificial Sequence
  primer
ctacatgttctttcttccct

SEQ ID NO: 149 - DNA, 20 bp, Artificial Sequence
  primer
cctcactcctcacatgttca

SEQ ID NO: 150 - DNA, 20 bp, Artificial Sequence
  primer
taaaccccaaaccccctttc

SEQ ID NO: 151 - DNA, 21 bp, Artificial Sequence
  primer
acaggaatgagagtaagaaag

SEQ ID NO: 152 - DNA, 20 bp, Artificial Sequence
  primer
gaggttgaggctacagtgag

SEQ ID NO: 153 - DNA, 20 bp, Artificial Sequence
  primer
cctctagaaagccaaccctc

SEQ ID NO: 154 - DNA, 19 bp, Artificial Sequence
  primer
ttcccacagtttacaaccc

SEQ ID NO: 155 - DNA, 19 bp, Artificial Sequence
  primer
gatctcactatgttgccca

SEQ ID NO: 156 - DNA, 20 bp, Artificial Sequence
  primer
gttttgtgctgacattggag

SEQ ID NO: 157 - DNA, 22 bp, Artificial Sequence
  primer
ctaccactttacttctcatcag

SEQ ID NO: 158 - DNA, 20 bp, Artificial Sequence
  primer
cacgttaaaaaacaaaagac

SEQ ID NO: 159 - DNA, 20 bp, Artificial Sequence
  primer
gaggaatgcagaatgttagc

SEQ ID NO: 160 - DNA, 20 bp, Artificial Sequence
  primer
aaaaggcaatggtgtgtatg

SEQ ID NO: 161 - DNA, 20 bp, Artificial Sequence
  primer
catttttcttttcgctggtc

SEQ ID NO: 162 - DNA, 20 bp, Artificial Sequence
  primer
ctgtggaatattgatgctat

SEQ ID NO: 163 - DNA, 20 bp, Artificial Sequence
  primer
tttgaggggacagctaggga

SEQ ID NO: 164 - DNA, 20 bp, Artificial Sequence
  primer
gtgactaagtgaaactggaa

SEQ ID NO: 165 - DNA, 20 bp, Artificial Sequence
  primer
catgcaactctcctttcaaa

SEQ ID NO: 166 - DNA, 20 bp, Artificial Sequence
  primer
cctcctatcttctttctcac

SEQ ID NO: 167 - DNA, 20 bp, Artificial Sequence
  primer
gtgaagaatagaggtagggt

SEQ ID NO: 168 - DNA, 20 bp, Artificial Sequence
  primer
gccaacctcattctactttt

SEQ ID NO: 169 - DNA, 20 bp, Artificial Sequence
  primer
gaattagaggataggcagca

SEQ ID NO: 170 - DNA, 20 bp, Artificial Sequence
  primer
cagaggtgataacagataca

SEQ ID NO: 171 - DNA, 20 bp, Artificial Sequence
  primer
gttcctgattgtgttggttt

SEQ ID NO: 172 - DNA, 21 bp, Artificial Sequence
  primer
acacataatcttaactccaag

SEQ ID NO: 173 - DNA, 20 bp, Artificial Sequence
  primer
ggtgacagagctttttagtg

SEQ ID NO: 174 - DNA, 20 bp, Artificial Sequence
  primer
tctttgtagttgctgtttgc

SEQ ID NO: 175 - DNA, 20 bp, Artificial Sequence
  primer
ggaaaagggggttgatatag

SEQ ID NO: 176 - DNA, 20 bp, Artificial Sequence
  primer
gggaaatgaaaagaggaaac

SEQ ID NO: 177 - DNA, 20 bp, Artificial Sequence
  primer
gcacatttctcttcagcaca

SEQ ID NO: 178 - DNA, 20 bp, Artificial Sequence
  primer
cttaagatgttccaggtgtg

SEQ ID NO: 179 - DNA, 20 bp, Artificial Sequence
  primer
ttaccgtttcaggtgtttgt

SEQ ID NO: 180 - DNA, 20 bp, Artificial Sequence
  primer
ggcctgcttctcctcagctt

SEQ ID NO: 181 - DNA, 20 bp, Artificial Sequence
  primer
gtgacgtaaagccgaacccg

SEQ ID NO: 182 - DNA, 20 bp, Artificial Sequence
  primer
ctaagggaacaaatggtgaa

SEQ ID NO: 183 - DNA, 20 bp, Artificial Sequence
  primer
tgagtgggtttacttgagtg